(19) United States
(12) Patent Application Publication
(10) Pub. No.: US 2008/0207683 A1
(43) Pub. Date: Aug. 28, 2008

(54) BIARYL-SUBSTITUTED TETRAHYDRO-PYRAZOLO-PYRIDINE MODULATORS OF CATHEPSIN S

(76) Inventors: Darin Allen, San Carlos, CA (US); Ingrid Choong, Los Altos, CA (US); Willard Lew, San Mateo, CA (US)

Correspondence Address: WOODCOCK WASHBURN LLP, CIRA CENTRE, 12TH FLOOR, 2929 ARCH STREET, PHILADELPHIA, PA, 19104, US

(21) Appl. No.: 12/031,410

(22) Filed: Feb. 14, 2008

Related U.S. Application Data
(60) Provisional application No. 60/889,979, filed on Feb. 15, 2007.

(51) Int. Cl. A61K 31/437; C07D 471/04; A61P 37/00; A61P 37/08; A61P 35/00

(52) U.S. Cl. 514/303; 546/119

(57) ABSTRACT

Biaryl-substituted tetrahydro-pyrazolo-pyridine compounds are described, which are useful as cathepsin S modulators. Such compounds may be used in pharmaceutical compositions and methods for the treatment of disease states, disorders, and conditions mediated by cathepsin S activity, such as psoriasis, pain, multiple sclerosis, atherosclerosis, and rheumatoid arthritis.

This application claims the benefit of U.S. provisional patent application Ser. No. 60/889,979, filed Feb. 15, 2007 which is incorporated herein by reference.

### FIELD

The present invention relates to certain biaryl-substituted tetrahydro-pyrazolo-pyridine compounds, pharmaceutical compositions containing them, and methods of using them for the treatment of disease states, disorders, and conditions mediated by cathepsin S activity.

### BACKGROUND

Cathepsin S is one of the major cysteine proteases expressed in the lysosome of antigen presenting cells, mainly dendritic cells, B cells and macrophages. Cathepsin S is best known for its critical function in the proteolytic digestion of the invariant chain chaperone molecules, thus controlling antigen presentation to CD4⁺ T cells by major histocompatibility complex class II molecules or to NK1.1⁺ T cells via CD1 molecules. Cathepsin S also appears to participate in direct processing of exogenous antigens for presentation by MHC class II to CD4⁺ T cells or crosspresentation by MHC class I molecules to CD8⁺ T cells. In addition, cathepsin S in secreted form is implicated in degradation of extracellular matrix, which may contribute to the pathology of a number of diseases, including arthritis, atherosclerosis, and chronic obstructive pulmonary disease. Therefore, inhibition of cathepsin S is a promising target for the development of novel therapeutics for a variety of indications. For a review, see: Thurmond, R. L. et al. Curr. Opin. Invest. Drugs 2005, 6(5), 473-482.

Pyrazole inhibitors of cathepsin S were disclosed in a series of applications from Ortho-McNeil, and publications on part of this work have appeared (See: Intl. Patent Appl. Publ. Nos. WO02/14314 (Feb. 21, 2002), WO02/14315 (Feb. 21, 2002), and WO02/14317 (Feb. 21, 2002). See also: Thurmond, R. L. et al. J. Pharm. Exp. Ther. 2004, 308, 268-276; and Thurmond, R. L. et al. J. Med. Chem. 2004, 47, 4799-4801). However, there remains a need for potent cathepsin S modulators with desirable pharmaceutical properties.

### SUMMARY

In one aspect the invention relates to compounds of the following Formula (I):

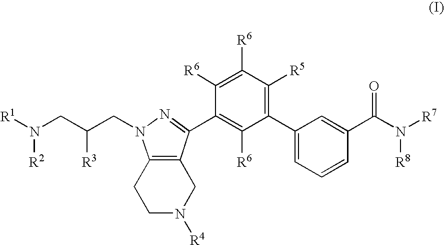

wherein:
R¹ and R² taken together with the nitrogen to which they are attached form a saturated monocyclic heterocycloalkyl group, optionally containing one additional heteroatom ring member that is O, S, or NR^{a}, and being unsubstituted or substituted with one, two, or three R^{b} substituents;
   where R^{a} is H, C₁₋₄alkyl, —COC₁₋₄alkyl, or —CO₂C₁₋₄alkyl;
   each R^{b} substituent is independently:
      i) OH, C₁₋₄alkyl, CF₃, NR^{c}R^{d}, —COC₁₋₄alkyl, —CO₂C₁₋₄alkyl; or —CONR^{e}R^{f};
      ii) a monocyclic heterocycloalkyl group unsubstituted or substituted with C₁₋₄alkyl, OH, —OC₁₋₄alkyl, NR^{c}R^{d}, or halo; or
      iii) a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
      where R^{c} is H or C₁₋₄alkyl;
      R^{d} is H, C₁₋₄alkyl, —COC₁₋₄alkyl, —CO₂C₁₋₄alkyl, or —CONR^{x}R^{y};
         where R^{x} and R^{y} are each independently H or C₁₋₄alkyl; and
      R^{e} and R^{f} are each independently H or C₁₋₄alkyl;
   or, alternatively, two R^{b} substituents at the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
R³ is H, OH, C₁₋₄alkyl, or —OC₁₋₄alkyl;
R⁴ is H, C₁₋₄alkyl, —COC₁₋₄alkyl, —SO₂C₁₋₄alkyl, —SO₂CF₃, —CONH₂, —CONHC₁₋₄alkyl, —CON(C₁₋₄alkyl)₂, —COCO₂C₁₋₄alkyl, —COCONH₂, or —COCONHC₁₋₄alkyl;
R⁵ is halo or CF₃;
each R⁶ is H or F;
R⁷ is H or C₁₋₆alkyl; and
R⁸ is Ar, —CH(Rⁱ)Ar,

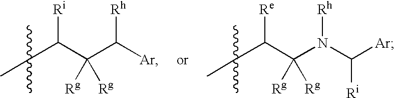

here each R^{g} is H or C₁₋₄alkyl, or two R^{g} groups together form a carbonyl;
each R^{h} is H or C₁₋₄alkyl;
Rⁱ is H or C₁₋₄alkyl; and
Ar is a phenyl, naphthyl, monocyclic heteroaryl, or bicyclic heteroaryl group, unsubstituted or substituted with one, two, or three R^{j} substituents; where each R^{j} substituent is independently selected from the group consisting of:
   C₁₋₄alkyl, monocyclic cycloalkyl, phenyl, —OC₁₋₄alkyl, —O—(CH₂)₀₋₁-(monocyclic cycloalkyl), halo, CF₃, —COC₁₋₄alkyl, —CO₂C₁₋₄alkyl, CO₂H, CN, NR^{r}R^{s}, —N(R^{r})COC₁₋₄alkyl, —N(R^{r})SO₂C₁₋₄alkyl, —NO₂, —SO₂C₁₋₄alkyl, —SO₂NR^{r}R^{s}, or —SO₃H, or two adjacent R^{j} substituents together form —(CH₂)₃—;
or, alternatively, an R^{j} substituent taken together with R^{h} forms —CH₂CH₂—;
where R^{r} and R^{s} are each independently H or C₁₋₄alkyl;
   and pharmaceutically acceptable salts, prodrugs, and metabolites thereof.

In certain embodiments, the compound of Formula (I) is a compound selected from those species described or exemplified in the detailed description below.

In a further aspect, the invention relates to pharmaceutical compositions each comprising: (a) an effective amount of at least one chemical entity selected from compounds of Formula (I), and pharmaceutically acceptable salts, prodrugs, and metabolites thereof; and (b) a pharmaceutically acceptable excipient.

In another aspect, the invention is directed to a method of treating a subject suffering from or diagnosed with a disease, disorder, or medical condition mediated by cathepsin S activity, comprising administering to the subject in need of such treatment an effective amount of at least one chemical entity selected from compounds of Formula (I), and pharmaceutically acceptable salts, prodrugs, and metabolites thereof. Diseases, disorders and medical conditions that are mediated by cathepsin S activity include those referred to herein.

Additional embodiments, features, and advantages of the invention will be apparent from the following detailed description and through practice of the invention.

### DETAILED DESCRIPTION

For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a saturated, straight- or branched-chain alkyl group having from 1 to 12 carbon atoms in the chain. Examples of alkyl groups include methyl (Me, which also may be structurally depicted by a bond, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

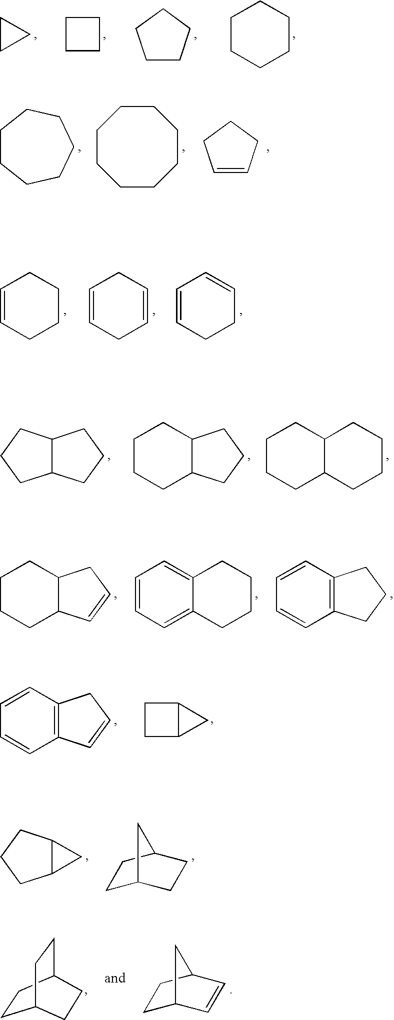

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative entities, in the form of properly bonded moieties, include:

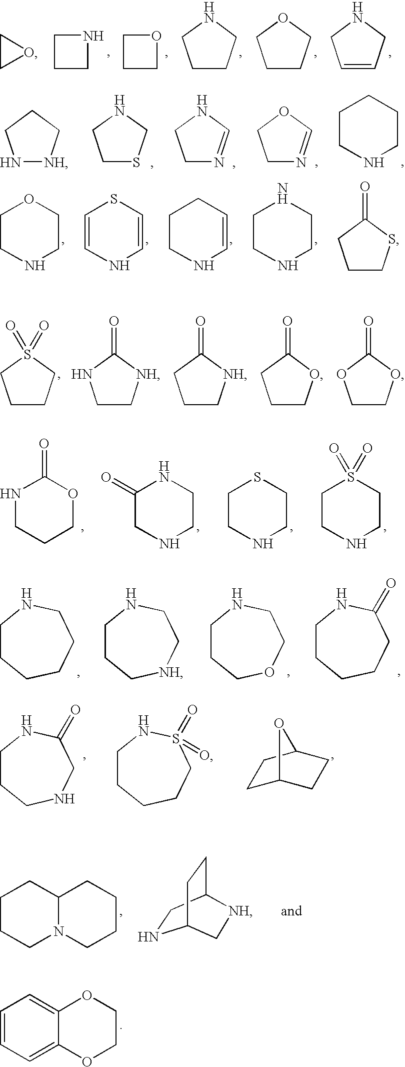

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

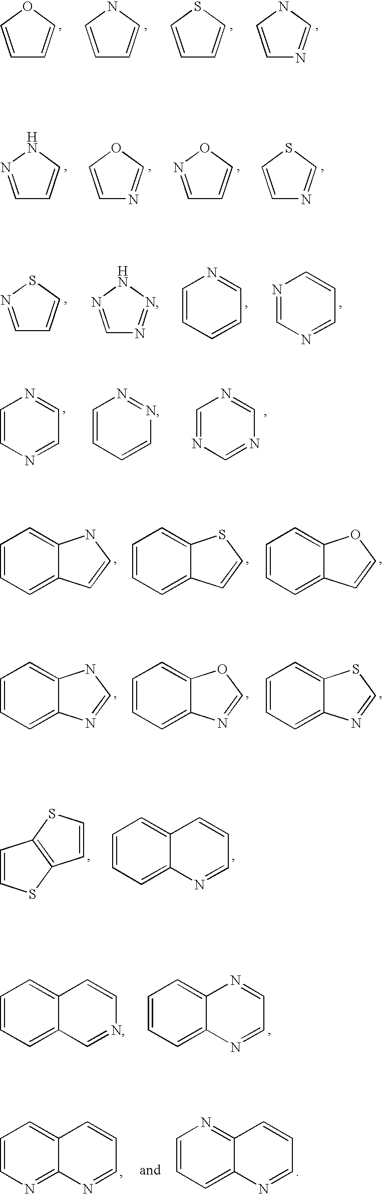

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system that yields a stable chemical structure.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, C¹⁵N, ¹⁸O, ¹⁷O, ³²P, ³³P, ³⁵S, ¹⁸F, ³⁶Cl, ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S²ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to any generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to any generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In some embodiments of Formula (I), —NR¹R² is a structure of Formula (II):

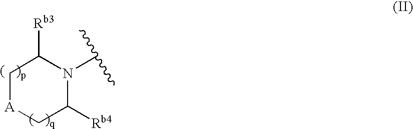

wherein:
A is NR^{a}, O, S, or C(R^{b1})(R^{b2});
   where R^{a} is H or C₁₋₄alkyl;
   R^{b1} is H, OH, or C₁₋₄alkyl; and
   R^{b2} is H; a monocyclic heterocycloalkyl group unsubstituted or substituted with C₁₋₄alkyl, OH, —OC₁₋₄alkyl, NR^{c}R^{d}, or halo; or a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
one of R^{b3} and R^{b4} is H and the other is C₁₋₄alkyl;
p is 0, 1, or 2; and
q is 0, 1, 2, or 3;
with the proviso that when A is NR^{a}, O, S, or SO₂, then p and q are each greater than or equal to 1.

In other embodiments, R¹ and R² taken together with the nitrogen to which they are attached form azetidine, pyrrolidine, piperidine, piperazine substituted with R^{a}, morpholine, or thiomorpholine, each unsubstituted or substituted with one, two, or three R^{b} substituents as described for Formula (I). In still other embodiments, R¹ and R² taken together with the nitrogen to which they are attached form pyrrolidine or piperidine, each unsubstituted or substituted with one, two, or three R^{b} substituents as described for Formula (I).

In some embodiments, R^{a} is H, methyl, isopropyl, acetyl, or tert-butoxycarbonyl.

In some embodiments, each R^{b} substituent is independently OH, methyl, propyl, CF₃, dimethylamino, acetamido, tert-butoxycarbamoyl, fluoro, or methoxy. In other embodiments, R^{b} is pyrrolidinyl, 2-oxo-pyrrolidinyl, or piperidinyl. In still other embodiments, R^{b} is pyrrolidin-1-yl or 2-oxo-pyrrolidin-1-yl.

In some embodiments, R³ is H or OH.

In some embodiments, R⁴ is H, methyl, —SO₂CH₃, acetyl, or tert-butoxycarbonyl. In other embodiments, R⁴ is —SO₂CH₃.

In some embodiments, R⁵ is chloro or CF₃. In other embodiments, R⁵ is chloro.

In some embodiments, R⁶ is H.

In some embodiments, R⁷ is H.

In some embodiments, R⁸ is Ar. In other embodiments, R³ is —CH(Rⁱ)Ar. In still other embodiments, R³ is —(CH₂)₂N(R^{h})Ar.

In some embodiments, each R^{g} is H or methyl. In other embodiments, two R^{g} groups together form a carbonyl.

In some embodiments, R^{h} is H or methyl.

In some embodiments, Rⁱ is H, methyl, or ethyl.

In some embodiments, Ar is a phenyl, naphthyl, pyridinyl, pyrimidinyl, oxazolyl, thiophenyl, thiazolyl, indanyl, indolyl, benzimidazolyl, or benzothiazolyl group, unsubstituted or substituted with one, two, or three R^{j} substituents. In still other embodiments, Ar is 4-methoxyphenyl, 4-methylphenyl, indan-4-yl, 3-chloro-4-methoxyphenyl, 4-cyclopentylmethoxy-phenyl, 6-methoxy-pyridin-3-yl, pyridin-3-yl, oxazol-2-yl, 1H-indol-2-yl, thiophen-2-yl, 5-methyl-1H-benzoimidazol-2-yl, 1H-benzoimidazol-2-yl, thiazol-2-yl, 5-chloro-1H-benzoimidazol-2-yl, 4-tert-butyl-thiazol-2-yl, 4-phenyl-thiazol-2-yl, 5-fluoro-benzothiazol-2-yl, benzothiazol-2-yl, 5,6-difluoro-1H-benzoimidazol-2-yl, 3,4-dimethyl-phenyl, or 4-isopropyl-phenyl.

In some embodiments, —N(R^{h})—Ar is 2,3-dihydro-indolyl (or “indanyl”), unsubstituted or substituted with one or two additional R^{j} substituents. In other embodiments, —N(R^{h})—Ar is 5-fluoro-2,3-dihydro-indol-1-yl, 7-dimethylsulfamoyl-2,3-dihydro-indol-1-yl, 6-dimethylsulfamoyl-2,3-dihydro-indol-1-yl, 6-fluoro-2,3-dihydro-indol-1-yl, or 5-methyl-2,3-dihydro-indol-1-yl.

In some embodiments, each R^{j} substituent is independently methyl, isopropyl, tert-butyl, cyclopentyl, phenyl, methoxy, isopropoxy, cyclopentylmethoxy, cyclohexyloxy, chloro, fluoro, CF₃, —NO₂, —SO₂N(CH₃)₂, or —SO₃H, or two adjacent R^{j} substituents together form —(CH₂)₃—. In other embodiments, an R^{j} substituent taken together with R^{h} forms —CH₂CH₂—.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), pharmaceutical compositions containing such pharmaceutically acceptable prodrugs, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “active agents”) of the present invention are useful in the methods of the invention. The active agents may be used in the inventive methods for the treatment or prevention of medical conditions, diseases, or disorders mediated through modulation of cathepsin S, such as those described herein. Symptoms or disease states are intended to be included within the scope of “medical conditions, disorders, or diseases.”

Accordingly, the invention relates to methods of using the active agents described herein to treat subjects diagnosed with or suffering from a disease, disorder, or condition mediated through cathepsin S activity, such as an autoimmune disease, an allergic condition, inflammation, a bowel disorder, tissue transplant rejection, pain, or cancer. Active agents according to the invention may therefore be used as immunomodulating agents, immunosuppressants, anti-allergy agents, anti-inflammatory agents, analgesics, or anti-cancer agents.

In some embodiments, an active agent of the present invention is administered to treat lupus, asthma, allergic reaction, atopic allergy, hay fever, atopic dermatitis, food allergy, rhinitis (such as allergic rhinitis and the inflammation caused by non-allergic rhinitis), skin immune system disorders (such as psoriasis), uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease (IBD), chronic obstructive pulmonary disorder (COPD), tissue transplant rejection, pain, chronic pain (such as pain due to conditions such as cancer, neuropathic pain, rheumatoid arthritis, osteoarthritis and inflammatory conditions), or cancer (and cancer-related processes such as angiogenesis, tumor growth, cell proliferation, and metastasis). In certain embodiments, an active agent of the present invention is administered to treat psoriasis, pain, multiple sclerosis, atherosclerosis, or rheumatoid arthritis.

Thus, the active agents may be used to treat subjects diagnosed with or suffering from a disease, disorder, or condition mediated through cathepsin S activity. The term “treat” or “treating” as used herein is intended to refer to administration of an active agent or composition of the invention to a subject for the purpose of effecting a therapeutic or prophylactic benefit through modulation of cathepsin S activity. Treating includes reversing, ameliorating, alleviating, inhibiting the progress of, lessening the severity of, or preventing a disease, disorder, or condition, or one or more symptoms of such disease, disorder or condition mediated through modulation of cathepsin S activity. The term “subject” refers to a mammalian patient in need of such treatment, such as a human. “Modulators” include both inhibitors and activators, where “inhibitors” refer to compounds that decrease, prevent, inactivate, desensitize or down-regulate cathepsin S expression or activity, and “activators” are compounds that increase, activate, facilitate, sensitize, or up-regulate cathepsin S expression or activity.

In treatment methods according to the invention, an effective amount of at least one active agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by cathepsin S activity, such as another cathepsin S modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, artisans will note that the various transformations described in the following Schemes may be performed in a different order than that depicted. Unless otherwise specified, the variables are as defined above in reference to Formula (I).

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| Ethyl acetate | EtOAc |
| tert-Butylcarbamoyl | BOC |
| Bovine serum albumin | BSA |
| Diethyl ether | Et₂O |
| N-Methyl morpholine | NMM |
| Diethyl azodicarboxylate | DEAD |
| Tris(dibenzylideneacetone) dipalladium | Pd₂dba₃ |
| High-pressure liquid chromatography | HPLC |
| Thin layer chromatography | TLC |
| Diisobutylaluminum hydride | DIBAL |
| Acetate | OAc |
| Acetic acid | AcOH |
| O-(7-Azabenzotriazol-1-yl)-N,N•N′,N′- | HATU |
| tetramethyluronium hexafluorophosphate | |
| Diisopropylethylamine | DIEA |
| 4-(Dimethylamino)pyridine | DMAP |
| 1-(3-Dimethylaminopropyl)-3- | EDC |
| ethylcarbodiimide hydrochloride | |
| 1-Hydroxybenzotriazole | HOBt |
| Methanesulfonyl chloride | MsCl |
| Tetrabutylammonium fluoride | TBAF |
| (Trimethylsilyl)acetylene | TMSA |
| Triethylamine | TEA |
| Hydroxysuccinimide | HOSu |
| 1,1′-bis(diphenylphosphino)ferrocene | Pd•dppf |
| palladium | |

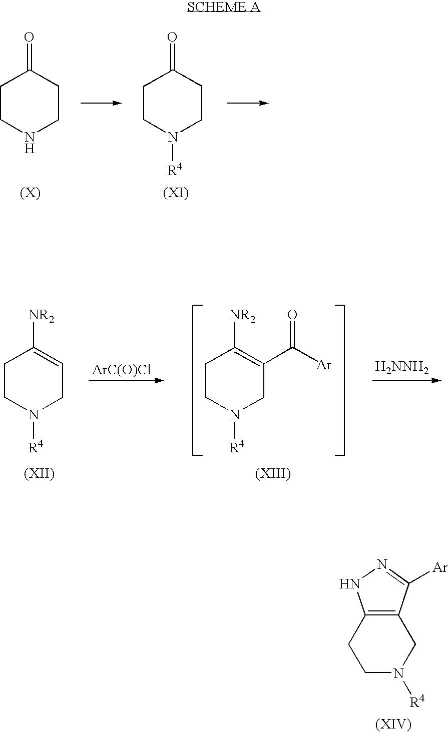

Referring to Scheme A, the tetrahydro-pyrazolo-pyridine core structure of Formula (I) may be prepared from commercially available piperidones (X). Installation of the R⁴ substituent is accomplished through, for example, alkylation, acylation, sulfonylation, amide formation, or other suitable methods known in the art to provide ketones (XI). Enamine formation according to general methods gives enamines (XII), which are then reacted with acyl chlorides, ArC(O)Cl, where Ar is a suitably substituted phenyl group, in the presence of a suitable tertiary amine base, to form enamines (XIII) (not isolated). In situ reaction of the enamines with hydrazine generates pyrazoles (XIV).

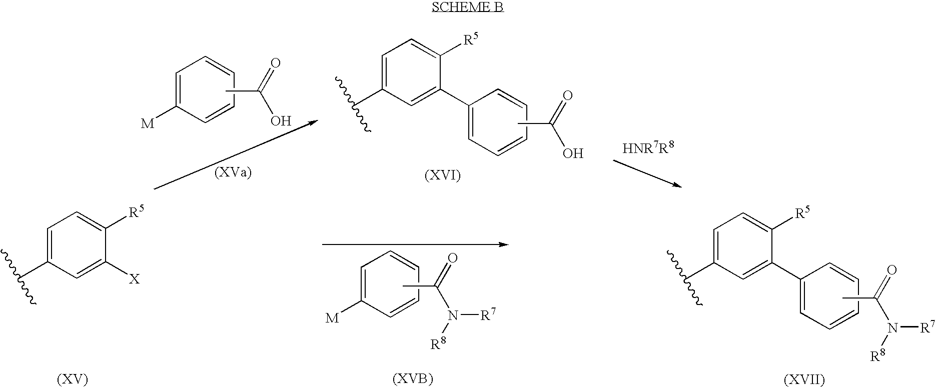

Where Ar is a suitably substituted group as in Formula (XV), where X is iodide or triflate, formation of biaryl acids (XVI) is accomplished palladium-mediated coupling with metallic reagents (XVa) or (XVb) such as boronic acids (where M is —B(OH)₂) or tin reagents (where M is Sn(alkyl)₃). Coupling with acids (XVa) yields biaryl acids (XVIa), which are converted into amides (XVII) by coupling with amines HNR⁷R⁸ using standard amide coupling methods known in the art. Amines HNR⁷R⁸ are commercially available or are prepared according to methods known in the art. In preferred embodiments, acids (XVI) are activated by coupling with HOSu, and the resulting succinamide analogs are reacted with amines HNR⁷R⁸. Alternatively, coupling of compounds (XV) with metal reagents (XVb) provide amides (XVII) directly.

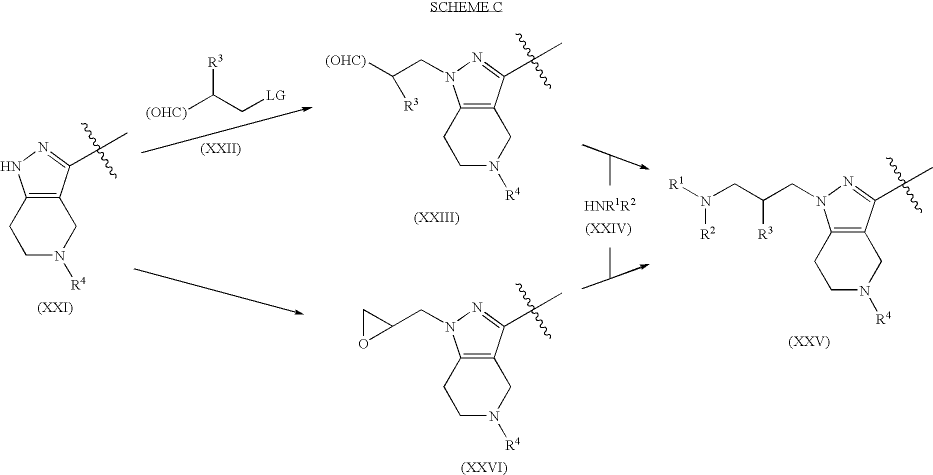

Two variations for the installation of the propyl amino chain are shown in Scheme C. Pyrazoles (XXI) are alkylated with optionally protected aldehydes (XXII), where R³ is H, C₁₋₄alkyl, or —OC₁₋₄alkyl, and LG is a suitable leaving group, such as a chloride, bromide, iodide, mesylate or tosylate, to give compounds (XXIII). If the aldehyde group is protected, for example, as an acetal, deprotection of (XXIII) is accomplished under general conditions. The resulting aldehydes are reacted with amines (XXIV) under reductive amination conditions, to provide propyl amines (XXV) where R³ is H, C₁₋₄alkyl, or —OC₁₋₄alkyl. Alternatively, pyrazoles (XXI) are reacted with epichlorohydrin, in the presence of a suitable base, to give epoxides (XXVI). Epoxide opening with amines (XXIV), preferably at elevated temperatures, yields propyl amines (XXV) where R³ is OH.

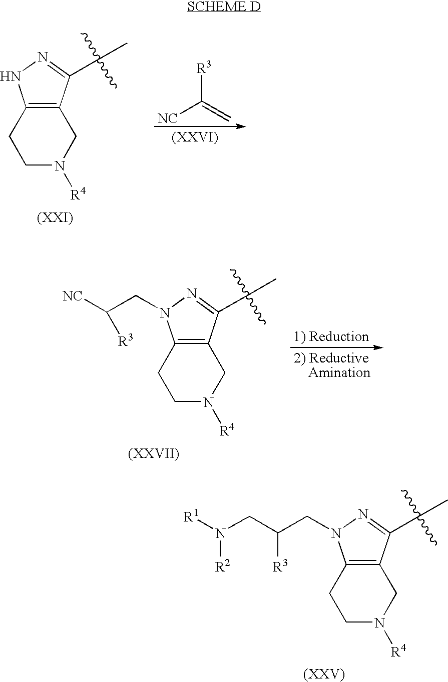

In another embodiment, addition of pyrazoles (XXI) to α,β-unsaturated nitriles (XXVI), in the presence of a suitable base, such as aq. NaOH, generates nitriles (XXVII). Reduction of the nitriles to the corresponding aldehydes (XXIII, not shown) is accomplished with a reducing agent such as DIBAL-H. Reductive amination of aldehydes (XXIII) with amines (XXIV) gives amines (XXV) as described in Scheme C.

Compounds of Formula (I) may be converted to their corresponding salts using methods described in the art. For example, an amine of Formula (I) may be treated with trifluoroacetic acid, HCl, or citric acid in a solvent such as Et₂O, CH₂Cl₂, THF, or MeOH to provide the corresponding salt form.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

### Chemistry

In obtaining the compounds described in the examples below and the corresponding analytical data, the following experimental and analytical protocols were followed unless otherwise indicated.

Unless otherwise stated, reaction mixtures were magnetically stirred at room temperature (rt). Where solutions are “dried,” they are generally dried over a drying agent such as Na₂SO₄ or MgSO₄. Where mixtures, solutions, and extracts were “concentrated”, they were typically concentrated on a rotary evaporator under reduced pressure.

Microwave reactions were performed on a Personal Chemistry Emrys Optimizer. Individual reactions were heated to the desired temperature and held at that temperature for the allotted time.

Analytical HPLC retention times are reported in minutes, and were obtained on an Agilent HP-1100 instrument with a Phenomenex Luna C-18 (5 uM, 4.6×150 mm) column, with a flow rate of 1 mL/min, detection at 230, 254, and 280 nM, and a gradient of 10 to 100% CH₃CN (0.05% TFA)/H₂O (0.05% TFA).

Preparatory HPLC purifications were typically performed on a Phenomenex Synergi column (4 μm, 21×150 mm), with a flow rate of 25 mL/min, and solvent conditions as described for Analytical HPLC.

Mass spectra (MS) were obtained on an Agilent series 1100 MSD using electrospray ionization (ESI) in positive mode unless otherwise indicated. Calculated (calcd.) mass corresponds to the exact mass. The MS data presented is the m/z found (typically [M+H]⁺) for the molecular ion.

Nuclear magnetic resonance (NMR) spectra were obtained on Bruker model DRX spectrometers (400, 500, or 600 MHz). NMR interpretation was performed using ACD Spec/Manager software to assign chemical shift and multiplicity. The format of the ¹H NMR data below is: chemical shift in ppm downfield of the tetramethylsilane reference (multiplicity, coupling constant J in Hz, integration). All ¹H NMR data was acquired in CD₃OD solvent unless otherwise indicated.

Chemical names were generated using ChemDraw Version 6.0.2 (CambridgeSoft, Cambridge, Mass.).

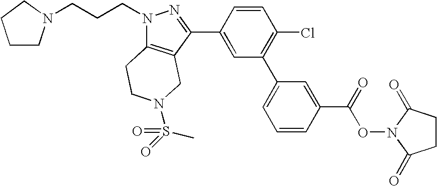

### Intermediate 1; 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 2,5-dioxo-pyrrolidin-1-yl ester

A. 1-Methanesulfonyl-piperidin-4-one. To a solution of 4-piperidone monohydrate hydrochloride (90 g, 586 mmol) in CHCl₃ (300 mL) and H₂O (300 mL) was added K₂CO₃ (324 g, 2340 mmol). The slurry was cooled to 0° C. and treated with MsCl (136 mL, 1.76 mmol) by dropwise addition over a 1 h period (gas evolution was observed). The reaction mixture was allowed to stir for 72 h and was partitioned between CH₂Cl₂ (500 mL) and aq. NaHCO₃ (500 mL). The aqueous layer was extracted with CH₂Cl₂ (3×200 mL). The organic layer was washed with 1% KHSO₄ (250 mL), dried over MgSO₄, and concentrated to give the desired product (90.5 g, 87%) as a white solid. HPLC: Rₜ=2.2. MS (ESI): mass calcd. for C₆H₁₁NO₃S, 178.1; m/z found, 178.1 [M+H]⁺.

B. 3-(4-Chloro-3-iodo-phenyl)-5-methanesulfonyl-4,5,6,7-tetrahydro-1H-pyrazolo[4,3-c]pyridine. To a solution of the above piperidone (10 g, 56 mmol) and p-toluenesulfonic acid (40 mg) in benzene (60 mL) was added morpholine (4.9 mL, 56 mmol). The reaction mixture was heated in a flask equipped with a condenser and a Dean-Stark trap at 90° C. for 16 h. The reaction mixture was cooled and concentrated to give the desired enamine as a beige solid, which was used without further purification. The enamine was dissolved in CH₂Cl₂ (40 mL), treated with TEA (9.4 mL, 67.2 mmol), and cooled to 0° C. To this solution was added 4-chloro-3-iodobenzoyl chloride* (16.9 g, 56 mmol). The reaction mixture was allowed to warm to rt, stirred for 14 h, and then concentrated. The resulting red oil was diluted with EtOH (56 mL) and treated with hydrazine (5.34 mL, 170 mmol) at 0° C. The resulting slurry was allowed to warm to rt and stirred for 16 h. EtOAc (120 mL) was added, and after 2 h the resulting precipitate was filtered and washed with additional EtOAc to afford the desired product as a white solid (8.80 g, 36%). HPLC: Rₜ=6.08. MS (ESI): mass calcd. for C₁₃H₁₃ClIN₃O₂S, 437.7; m/z found, 438.1 [M+H]⁺. ¹H NMR (DMSO-d₆): 8.05 (d, J=1.9, 1H), 7.51 (d, J=8.3, 1H), 7.43 (dd, J=8.4, 1.9, 2H), 4.30 (s, 2H), 3.36 (t, J=5.8, 2H), 3.30 (br s, 1H), 2.86 (s, 3H), 2.69 (t, J=5.6, 2H).

*Prepared by dissolving 4-chloro-3-iodobenzoic acid (15.8 g, 56 mmol) in CH₂Cl₂ (40 mL) and treating with oxalyl chloride (4.1 mL, 46.7 mmol) and a catalytic amount of DMF (0.40 mL; vigorous gas evolution). The mixture was stirred at rt for 3 h. The reaction mixture was concentrated to afford a white solid, which was used without further purification.

C. 3-(4-Chloro-3-iodo-phenyl)-1-(2-[1,3]dioxolan-2-yl-ethyl)-5-methanesulfonyl-4,5,6,7-tetrahydro-1H-pyrazolo[4,3-c]pyridine. A slurry of the above pyrazole (10 g, 22.8 mmol) and Cs₂CO₃ (11.9 g, 45.6 mmol) in DMF (75 mL) was stirred at rt for 2 h. 2-(2-Bromoethyl)1,3-dioxolane (3.5 mL, 34.2 mmol) was added dropwise and stirring maintained for 12 h. Ice H₂O was added slowly to form a precipitate. The white solid was collected by suction filtration and washed with H₂O and Et₂O to afford the desired product (10.4 g, 85%). HPLC: Rₜ=6.98. MS (ESI): mass calcd. for C₁₈H₂₁ClIN₃O₄S, 537.8; m/z found, 538.2 [M+H]⁺. ¹H NMR (CDCl₃): 8.15 (s, 1H), 7.46-7.45 (m, 2H), 4.83 (t, J=4.6, 1H), 4.49 (s, 2H), 4.17 (t, J=7.1, 2H), 4.01-3.97 (m, 2H), 3.89-3.86 (m, 2H), 3.65 (t, J=5.8, 2H), 2.89 (s, 3H), 2.87 (t, J=5.8, 2H), 2.28-2.26 (m, 2H).

D. 3-(4-Chloro-3-iodo-phenyl)-5-methanesulfonyl-1-(3-morpholin-4-yl-propyl)-4,5,6,7-tetrahydro-1H-pyrazolo[4,3-c]pyridine. A mixture of the above acetal (4.65 g, 8.64 mmol) and 1 N HCl (19 mL) in acetone (75 mL) was heated at 55° C. for 5 h. The clear solution was diluted with CH₂Cl₂ and washed with saturated aqueous (satd. aq.) NaHCO₃. The combined organic extracts were dried (Na₂SO₄), filtered, and concentrated to give a white solid, which was used directly in the next reaction. The crude aldehyde was dissolved in CH₂Cl₂ (80 mL) and pyrrolidine (2.5 mL, 17.3 mmol) and acetic acid (1.0 mL) were added sequentially. After 10 min, NaBH(OAc)₃ (3.48 g, 13 mmol) was added and stirring was continued for 2 h. After the addition of satd. aq. NaHCO₃, the layers were separated and the aqueous layer was extracted with CH₂Cl₂ (3×). The combined organic extracts were washed with brine, dried (MgSO₄), filtered, and concentrated to give an orange oil.

E. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid. To a solution of the above aryl iodide (6 g, 11.2 mmol), 3-carboxyphenyl boronic acid (2 g, 12.3 mmol), Pd-dppf (0.914 g, 1.1 mmol) in DMF (35 mL) was added 2 M aq. K₂CO₃ (11.2 mL). The reaction mixture was stirred under N₂ at 90° C. for 3 h. Upon cooling to rt, the mixture was diluted with CH₂Cl₂ and a black precipitate resulted. The solution was decanted away and the desired product precipitate was collected. HPLC: Rₜ=1.494. MS (ESI): mass calcd. for C₂₇H₃₁ClN₄O₄S, 542.2; m/z found, 543.1 [M+H]⁺.

F. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 2,5-dioxo-pyrrolidin-1-yl ester. A solution of the above carboxylic acid (1.6 g, 2.9 mmol) was dissolved in DMF (10 mL) and CH₂Cl₂ (20 mL) was treated with hydroxysuccinimide (0.366 g, 3.2 mmol), HATU (1.3 g, 3.5 mmol), and DIEA (1 mL, 5.8 mmol). After stirring for 2 h, the mixture was diluted with CH₂Cl₂ (50 mL) and washed with satd. aq. NaHCO₃ (20 mL). The organic layer was dried over MgSO₄ and concentrated to give the desired product. HPLC: Rₜ=1.657. MS (ESI): mass calcd. for C₃₁H₃₄ClN₅O₆S, 639.2; m/z found, 640.1 [M+H]⁺.

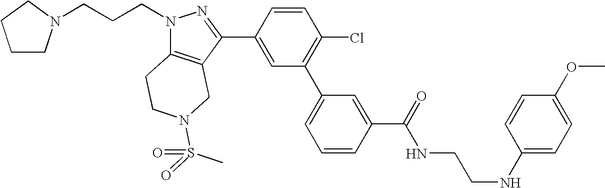

### Example 1

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-methoxy-phenylamino)-ethyl]-amide

A. N¹-(4-Methoxy-phenyl)-ethane-1,2-diamine. A solution of 2-oxazolidinone (1.5 g, 17.2 mmol) in 2-(2-ethoxyethoxy)ethanol (20 mL) was treated with p-anisidine hydrochloride (2.1 g, 17.2 mmol) and heated in the microwave at 150° C. at 300 W for 10 min. The solution was cooled to rt and Et₂O (50 mL) was added. The resulting solid precipitate was filtered and washed with Et₂O (3×20 mL). HPLC: Rₜ=0.23. MS (ESI): mass calcd. for C₉H₁₄N₂O, 166.1; m/z found, 150 [M-16]⁺.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-methoxy-phenylamino)-ethyl]-amide. To a solution of Intermediate 1 (0.100 g, 0.2 mmol) and DIEA (0.1 mL, 0.6 mmol) in DMF (1 mL) was added N¹-(4-methoxy-phenyl)-ethane-1,2-diamine (0.029 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.425. MS (ESI): mass calcd. for C₃₆H₄₃ClN₆O₄S, 690.3; m/z found, 691.3 [M+H]⁺.

The compounds in Examples 2-4 were prepared according to the methods described for Example 1, substituting the appropriate amine for p-anisidine in Example 1, Step A.

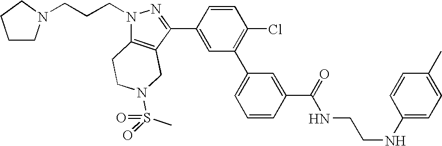

### Example 2

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (2-p-tolylamino-ethyl)-amide

HPLC: Rₜ=1.600. MS (ESI): mass calcd. for C₃₆H₄₃ClN₆O₃S, 674.3; m/z found, 675.3 [M+H]⁺.

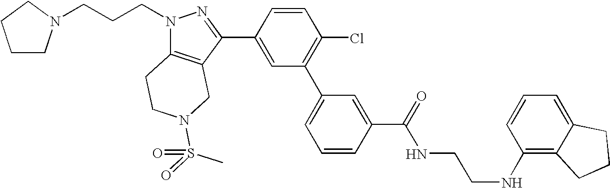

### Example 3

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(indan-4-ylamino)-ethyl]-amide

HPLC: Rₜ=1.592. MS (ESI): mass calcd. for C₃₈H₄₅ClN₆O₃S, 700.3; m/z found, 701.3 [M+H]⁺.

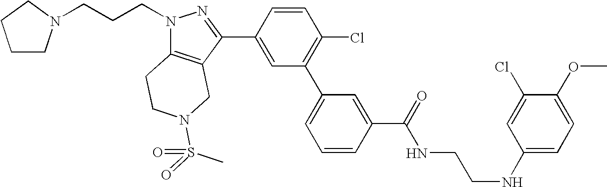

### Example 4

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-chloro-4-methoxy-phenylamino)-ethyl]-amide

HPLC: Rₜ=1.754. MS (ESI): mass calcd. for C₃₆H₄₂Cl₂N₆O₄S, 724.2; m/z found, 725.3 [M+H]⁺.

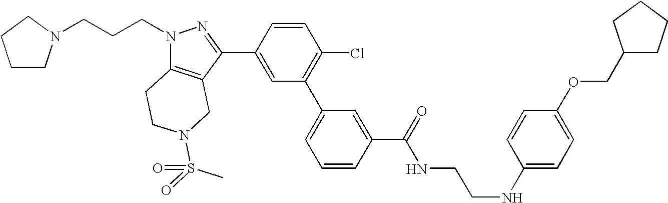

### Example 5

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-cyclopentylmethoxy-phenylamino)-ethyl]-amide

A. 1-Bromo-4-cyclopentylmethoxy-benzene. To a 0° C. solution of 4-bromophenol (2.56 g, 14.8 mmol), Ph₃P (4 g, 14.8 mmol), and cyclopentyl-methanol (1.6 mL, 14.8 mmol) in THF (50 mL) was added DEAD (2.5 mL, 14.8 mmol) dropwise. The reaction solution was allowed to warm to rt and stirred for 12 h. The mixture was concentrated and the resulting residue was diluted in hexanes until a white precipitate formed. The precipitate was removed by filtration and the filtrate was concentrated. Purification (SiO₂; 25% EtOAc in hexanes) provided the desired product (2.84 g, 75.1%). HPLC: Rₜ=2.35.

B. N¹-(4-Cyclopentylmethoxy-phenyl)-ethane-1,2-diamine. A solution of the above aryl bromide (1.4 g, 5.5 mmol) in 1,2-diaminoethane (1.5 mL, 22 mmol) was treated with CuSO₄ (0.175 g, 1.1 mmol) and heated in the microwave at 150° C. at 300 W for 20 min. The solution was diluted with EtOAc (20 mL) and washed with H₂O (3×20 mL). The organic layer was separated, dried over MgSO₄, and concentrated. HPLC: Rₜ=2.32. MS (ESI): mass calcd. for C₁₄H₂₂N₂O, 234.2; m/z found, 235.1 [M+H]⁺.

C. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-cyclopentylmethoxy-phenylamino)-ethyl]-amide. To a solution of Intermediate 1 (0.100 g, 0.2 mmol) and DIEA (0.1 mL, 0.6 mmol) in DMF (1 mL) was added the above amine (0.051 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.629. MS (ESI): mass calcd. for C₄₁H₅₁ClN₆O₄S, 758.3; m/z found, 759.3 [M+H]⁺.

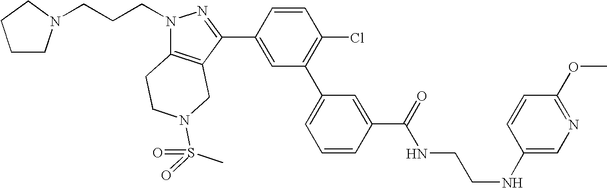

### Example 6

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-methoxy-pyridin-3-ylamino)-ethyl]-amide

A. N¹-(6-Methoxy-pyridin-3-yl)-ethane-1,2-diamine. A solution of 5-bromo-2-methoxy-pyridine (0.2 g, 0.1 mmol) in 1,2-diaminoethane (0.5 mL) was treated with CuSO₄ and heated in the microwave at 150° C. at 300 W for 10 min. The solution was diluted with EtOAc (10 mL) and washed with H₂O (3×10 mL). The organic layer was separated, dried over MgSO₄, and concentrated. HPLC: Rₜ=0.257. MS (ESI): mass calcd. for C₈H₁₃N₃O, 167.1; m/z found, 168 [M+H]⁺.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-methoxy-pyridin-3-ylamino)-ethyl]-amide. To a solution of Intermediate 1 (0.100 g, 0.2 mmol) and DIEA (0.1 mL, 0.6 mmol) in DMF (1 mL) was added the above amine (0.051 g, 0.2 mmol). After stirring for 2 h at rt, the mixture was concentrated to give a black oil. Purification via preparatory reverse phase HPLC followed by lyophilization afforded the title compound as an off-white solid. MS (ESI): mass calcd. for C₃₅H₄₂ClN₇O₄S 691.27; m/z found, 692.3 [M+H]⁺.

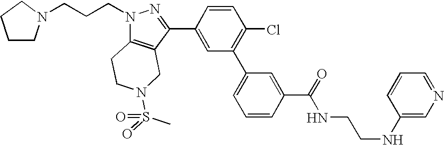

### Example 7

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(pyridin-3-ylamino)-ethyl]-amide

A. N¹-Pyridin-3-yl-ethane-1,2-diamine. A solution of 3-bromo-pyridine (0.2 g, 0.1 mmol) in 1,2-diaminoethane (0.5 mL) was treated with CuSO₄ and heated in the microwave at 150° C. at 300 W for 10 min. The solution was diluted with EtOAc (10 mL) and washed with H₂O (3×10 mL). The organic layer was separated, dried over MgSO₄ and the solution was concentrated. HPLC: Rₜ=0.227. MS (ESI): mass calcd. for C₇H₁₁N₃, 137.1; m/z found, 138 [M+H]⁺.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(pyridin-3-ylamino)-ethyl]-amide. To a solution of Intermediate 1 (0.100 g, 0.2 mmol) and DIEA (0.1 mL, 0.6 mmol) in DMF (1 mL) was added the above amine (0.051 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.423. MS (ESI): mass calcd. for C₃₄H₄₀ClN₇O₃S, 661.3; m/z found, 662.3 [M+H]⁺.

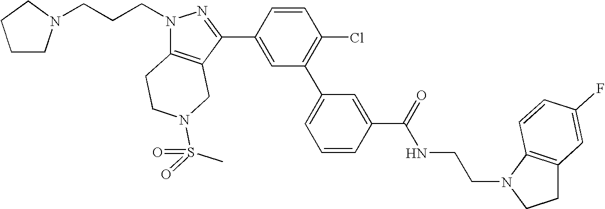

### Example 8

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-ethyl]-amide

A. 2-(5-Fluoro-2,3-dihydro-indol-1-yl)-ethylamine. To a solution of 5-fluoro-2,3-dihydro-(1H)-indole (0.196 g, 1.43 mmol) in MeOH (20 mL) was added N-Boc-glycine (0.250 mL, 1.57 mmol). After stirring for 10 min, a solution of NaBH₃CN (1 M in THF, 0.650 mL, 3.14 mmol) and 3 drops of acetic acid were added. After stirring for 12 h, the solution was diluted with EtOAc (20 mL) and washed with satd. aq. NaHCO₃ (3×20 mL). The organic layer was separated, dried over MgSO₄, and concentrated. To a solution of the resulting residue in dioxane (2 mL) was added 2 N HCl in Et₂O (4 mL). After stirring for 1 h, the mixture was concentrated to yield the crude product, which was used in the next step without further purification. HPLC: Rₜ=0.967. MS (ESI): mass calcd. for C₁₀H₁₃FN₂, 180.1; m/z found, 181.2 [M+H]⁺.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-ethyl]-amide. To a solution of Intermediate 1 (0.100 g, 0.2 mmol) and DIEA (0.1 mL, 0.6 mmol) in DMF (1 mL) was added 2-(5-fluoro-2,3-dihydro-indol-1-yl)-ethylamine (0.040 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.571. MS (ESI): mass calcd. for C₃₇H₄₂ClFN₆O₃S, 704.3; m/z found, 705.3 [M+H]⁺.

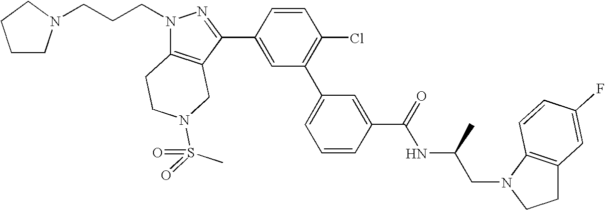

### Example 9

### (S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-1-methyl-ethyl]-amide

The title compound was prepared according to the method described for Example 8, substituting N-Boc-alanine for N-Boc-glycine in Example 8, Step A. HPLC: Rₜ=1.726. MS (ESI): mass calcd. for C₃₈H₄₄ClFN₆O₃S, 718.3; m/z found, 719.3 [M+H]⁺.

The compounds in Examples 10-11 were prepared according to the method described for Example 8, substituting the appropriate indole for 5-fluoro-2,3-dihydro-(1H)-indole in Example 8, Step A.

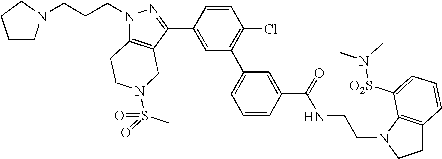

### Example 10

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(7-dimethylsulfamoyl-2,3-dihydro-indol-1-yl)-ethyl]-amide

HPLC: Rₜ=1.617. MS (ESI): mass calcd. for C₃₉H₄₈ClN₇O₅S₂, 793.3; m/z found, 794.3 [M+H]⁺.

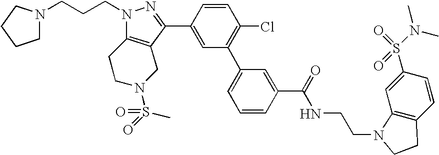

### Example 11

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-dimethylsulfamoyl-2,3-dihydro-indol-1-yl)-ethyl]-amide

HPLC: Rₜ=1.640. MS (ESI): mass calcd. for C₃₉H₄₈ClN₇O₅S₂, 793.3; m/z found, 794.3 [M+H]⁺.

The compounds in Examples 12-19 were prepared according to the method described for Example 1, omitting Example 1, Step A, and substituting the appropriate amine for N¹-(4-methoxy-phenyl)-ethane-1,2-diamine in Example 1, Step B.

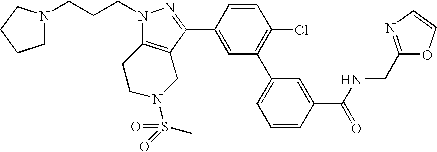

### Example 12

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (oxazol-2-ylmethyl)-amide

HPLC: Rₜ=1.431. MS (ESI): mass calcd. for C₃₁H₃₅ClN₆O₄S, 622.2; m/z found, 623.2 [M+H]⁺.

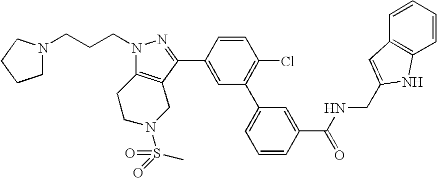

### Example 13

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (1H-indol-2-ylmethyl)-amide

HPLC: Rₜ=1.710. MS (ESI): mass calcd. for C₃₆H₃₉ClN₆O₃S, 670.3; m/z found, 671.3 [M+H]⁺.

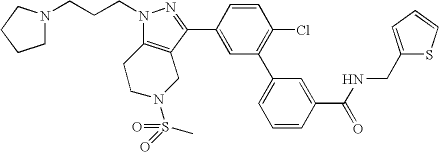

### Example 14

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiophen-2-ylmethyl)-amide

HPLC: Rₜ=1.621. MS (ESI): mass calcd. for C₃₂H₃₆ClN₅O₃S₂, 637.2; m/z found, 638.2 [M+H]⁺.

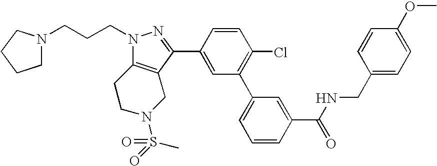

### Example 15

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 4-methoxy-benzylamide

HPLC: Rₜ=1.640. MS (ESI): mass calcd. for C₃₅H₄₀ClN₅O₄S, 661.3; m/z found, 662.3 [M+H]⁺.

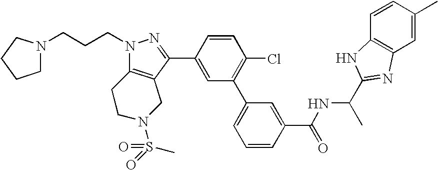

### Example 16

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide

HPLC: Rₜ=1.453. MS (ESI): mass calcd. for C₃₇H₄₂ClN₇O₃S, 699.3; m/z found, 700.3 [M+H]⁺.

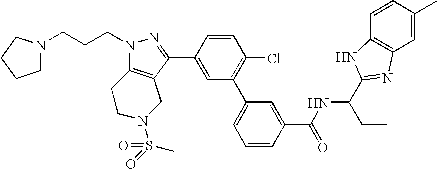

### Example 17

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-propyl]-amide

HPLC: Rₜ=1.491. MS (ESI): mass calcd. for C₃₈H₄₄ClN₇O₃S, 713.3; m/z found, 714.4 [M+H]⁺.

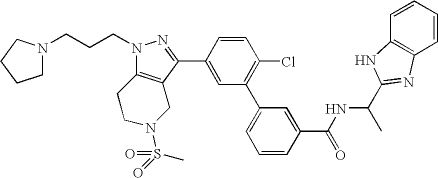

### Example 18

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(1H-benzoimidazol-2-yl)-ethyl]-amide

HPLC: Rₜ=1.405. MS (ESI): mass calcd. for C₃₆H₄₀ClN₇O₃S, 685.3; m/z found, 686.3 [M+H]⁺.

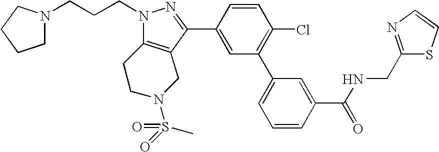

### Example 19

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiazol-2-ylmethyl)-amide

HPLC: Rₜ=1.481. MS (ESI): mass calcd. for C₃₁H₃₅ClN₆O₃S₂, 638.2; m/z found, 639.2 [M+H]⁺.

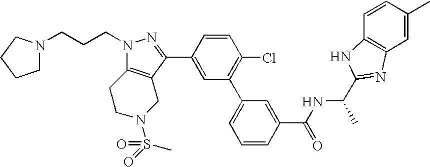

### Example 20

### (S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide

A. [1-(5-Methyl-1H-benzoimidazol-2-yl)-ethyl]-carbamic acid tert-butyl ester. To a solution of N-Boc-Ala-OH (1.9 g, 10 mmol) at 0° C. in DMF (50 mL) was added NMM (1.1 mL, 19 mmol). After stirring for 10 min, 4-methyl-benzene-1,2-diamine (1.2 g, 10 mmol) in DMF (5 mL) was added. After stirring at rt for 2 h, the mixture was concentrated and the resulting solid was dissolved in EtOAc (50 mL) and washed with satd. aq. NaHCO₃ (3×50 mL). The organic layer was separated, dried over MgSO₄, and concentrated. The crude mono-acylated amine was dissolved in acetic acid (30 mL) and heated to 70° C. for 1 h. The solution was concentrated, and the resulting cyclized intermediate was dissolved in EtOAc (50 mL) and washed with satd. aq. NaHCO₃ (3×50 mL). The organic layer was dried over MgSO₄ and concentrated to give the desired product.

B. 1-(5-Methyl-1H-benzoimidazol-2-yl)-ethylamine. The above intermediate was dissolved in TFA (30 mL) and stirred for 1 h. The mixture was concentrated to give the crude product, which was used in the next step without further purification.

C. (S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide. To a solution of Intermediate 1 (0.100 g, 0.2 mmol) and DIEA (0.1 mL, 0.6 mmol) in DMF (1 mL) was added 1-(5-methyl-1H-benzoimidazol-2-yl)-ethylamine (0.039 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.461. MS (ESI): mass calcd. for C₃₇H₄₂ClN₇O₃S, 699.3; m/z found, 700.3 [M+H]⁺.

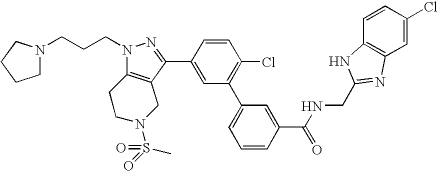

### Example 21

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-chloro-1H-benzoimidazol-2-ylmethyl)-amide

The title compound was prepared according to the methods described for Example 20, substituting N-Boc-Gly-OH for N-Boc-Ala-OH in Example 20, Step A. HPLC: Rₜ=1.457. MS (ESI): mass calcd. for C₃₅H₃₇Cl₂N₇O₃S, 705.2; m/z found, 706.2 [M+H]⁺.

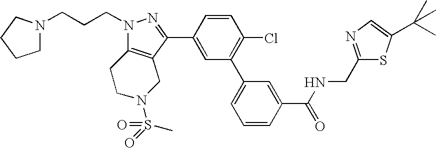

### Example 22

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-tert-butyl-thiazol-2-ylmethyl)-amide

A. Thiocarbamoylmethyl carbamic acid tert-butyl ester. To a solution of Boc-Gly-NH₂ (11 g, 62.9 mmol) in CH₂Cl₂ (300 mL) was added Lawesson's Reagent (13.2 g, 32.7 mmol). After stirring at rt for 12 h, the mixture was concentrated. Purification of the residue (SiO₂; 50% Et₂O in hexanes) provided the desired product (5.0 g, 42%).

B. (4-tert-Butyl-thiazol-2-ylmethyl)-carbamic acid tert-butyl ester. To a solution of the above compound (0.2 g, 1.05 mmol) in EtOH (5 mL) was added 1-bromo-3,3-dimethyl-butan-2-one (0.188 g, 1.05 mmol). After stirring 12 h at rt, the mixture was concentrated. Purification of the residue (SiO₂; 20% EtOAc in hexanes) provided the desired product. HPLC: Rₜ=1.79. MS (ESI): mass calcd. for C₁₃H₂₂N₂O₂S, 270.1; m/z found, 271.1 [M+H]⁺.

C. (4-tert-Butyl-thiazol-2-yl)-methylamine. The above intermediate was dissolved in TFA (30 mL) and stirred for 1 h. The mixture was concentrated to provide the crude product, which was used in the next step without further purification. HPLC: Rₜ=0.402. MS (ESI): mass calcd. for C₈H₁₄N₂S, 170.1; m/z found, 171.1 [M+H]⁺.

D. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-tert-butyl-thiazol-2-ylmethyl)-amide. To a solution of Intermediate 1 (0.100 g, 0.2 mmol) and DIEA (0.1 mL, 0.6 mmol) in DMF (1 mL) was added (4-tert-butyl-thiazol-2-yl)-methylamine (0.037 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.699. MS (ESI): mass calcd. for C₃₅H₄₃ClN₆O₃S₂, 694.3; m/z found, 695.3 [M+H]⁺.

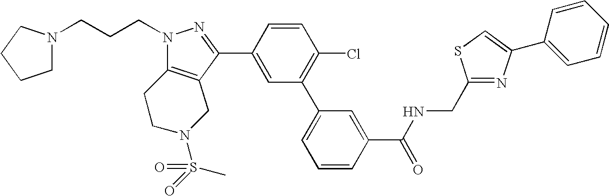

### Example 23

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-phenyl-thiazol-2-ylmethyl)-amide

The title compound was prepared according to the methods described for Example 22, substituting 2-bromo-1-phenyl-ethanone for 1-bromo-3,3-dimethyl-butan-2-one in Example 22, Step B. HPLC: Rₜ=1.583. MS (ESI): mass calcd. for C₃₇H₃₉ClN₆O₃S₂, 714.2; m/z found, 714.3 [M]⁺.

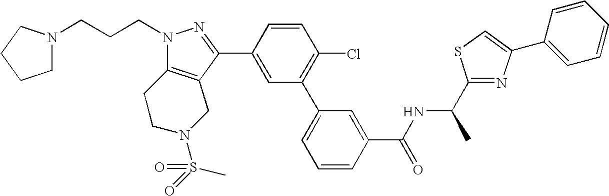

### Example 24

### (R)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(4-phenyl-thiazol-2-yl)-ethyl]-amide

The title compound was prepared according to the methods described in Example 23, substituting N-Boc-D-Ala-NH₂ for N-Boc-Gly-NH₂ in Step A. HPLC: Rₜ=1.753. MS (ESI): mass calcd. for C₃₈H₄₁ClN₆O₃S₂, 728.2; m/z found, 729.2 [M+H]⁺.

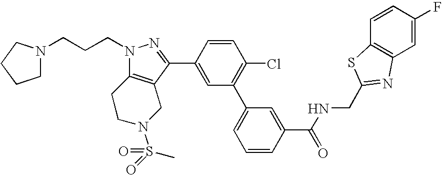

### Example 25

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-fluoro-benzothiazol-2-ylmethyl)-amide

A. (5-Fluoro-benzothiazol-2-ylmethyl)-carbamic acid tert-butyl ester. A solution of thiocarbamoyl-acetic acid tert-butyl ester (0.2 g, 1.05 mmol), 4-fluoro-2-iodo-phenylamine (0.230 g, 1.05 mmol), Pd₂ dba₃ (0.096 g, 0.105 mmol), and dppf (0.117 g, 0.210 mmol) in DMF (10 mL) was heated at 60° C. for 12 h. The mixture was diluted with EtOAc (20 mL) and washed with stad. aq. NaHCO₃ (3×20 mL). The organic layer was dried over MgSO₄ and concentrated. Purification of the residue (SiO₂; 20% EtOAc in hexanes) provided the desired product (0.216 g, 78%).

B. (5-Fluoro-benzothiazol-2-yl)-methylamine. A solution of the above intermediate in TFA (30 mL) was stirred for 1 h. The mixture was concentrated to give the crude product, which was used in the next step without further purification.

C. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-fluoro-benzothiazol-2-ylmethyl)-amide. To a solution of Intermediate 1 (0.100 g, 0.2 mmol) and DIEA (0.1 mL, 0.6 mmol) in DMF (1 mL) was added (5-fluoro-benzothiazol-2-yl)-methylamine (0.037 g, 0.2 mmol). After stirring for 2 h at rt, the mixture was concentrated to give a black oil. Purification via preparatory reverse phase HPLC followed by lyophilization afforded the desired product as an off-white solid. MS (ESI): mass calcd. for C₃₅H₃₆ClFN₆O₃S₂, 706.2; m/z found, 707.2 [M+H]⁺.

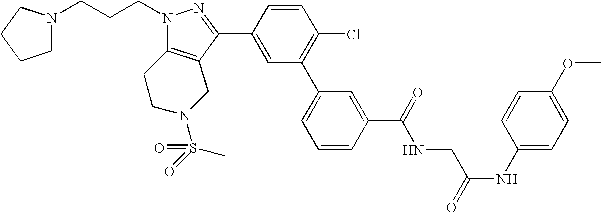

### Example 26

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [(4-methoxy-phenylcarbamoyl)-methyl]-amide

A. 2-Amino-N-(4-methoxy-phenyl)-acetamide. A solution of p-anisidine (0.25 g, 2.0 mmol), N-Boc-Gly-OH (0.26 g, 2.2 mmol), and HATU (0.926 g, 2.4 mmol) in CH₂Cl₂ (10 mL) was treated with DIEA (0.653 mL, 4 mmol), and stirred for 2 h. The mixture was diluted with CH₂Cl₂ (20 mL) and washed with 1 M HCl (2×10 mL). The organic layer was dried over MgSO₄ and concentrated. The resulting solid was dissolved in dioxane (10 mL) and treated with 4 N HCl (20 mL) and stirred for 1 h. Removal of solvent gave the desired product which was used in the next step without further purification. HPLC: Rₜ=0.293. MS (ESI): mass calcd. for C₉H₁₂N₂O₂, 180.1; m/z found, 181 [M+H]⁺.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [(4-methoxy-phenylcarbamoyl)-methyl]-amide. To a solution of Intermediate 1 (0.100 g, 0.2 mmol) and DIEA (0.1 mL, 0.6 mmol) in DMF (1 mL) was added the above amine (0.039 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.651. MS (ESI): mass calcd. for C₃₆H₄₁ClN₆O₅S, 704.3; m/z found, 705.3 [M+H]⁺.

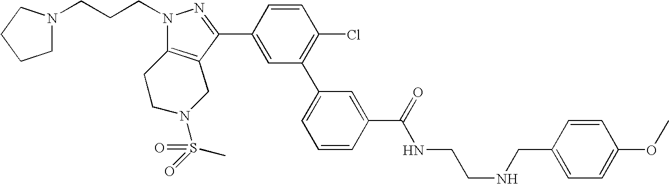

### Example 27

### 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-methoxy-benzylamino)-ethyl]-amide

A. N¹-(3-Methoxy-benzyl)-ethane-1,2-diamine. A solution of N-Boc-glycinal (0.1 g, 0.5 mmol), 3-methoxy benzylamine (0.076 g, 0.5 mmol), and NaBH(OAc)₃ (0.117 g, 0.6 mmol) in 1,2-dichloroethane (10 mL) was treated with AcOH (2 drops) and stirred for 1 h. The mixture was diluted with CH₂Cl₂ (10 mL) and washed with 1 M HCl (1×10 mL). The organic layer was dried over MgSO₄ and concentrated. The resulting solid was dissolved in dioxane (10 mL), treated with 4 N HCl (10 mL), and stirred for 1 h. Removal of solvent resulted in the desired product which was used in the next step without further purification.

B. 2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-methoxy-benzylamino)-ethyl]-amide. To a solution of Intermediate 1 (0.100 g, 0.2 mmol) and DIEA (0.1 mL, 0.6 mmol) in DMF (1 mL) was added the above amine (0.039 g, 0.2 mmol). After stirring for 2 h at rt, the mixture was concentrated to give a black oil. Purification via preparatory reverse phase HPLC followed by lyophilization afforded the title compound as an off-white solid. HPLC: Rₜ=1.554. MS (ESI): mass calcd. for C₃₇H₄₅ClN₆O₄S, 704.3; m/z found, 705.3 [M+H]⁺.

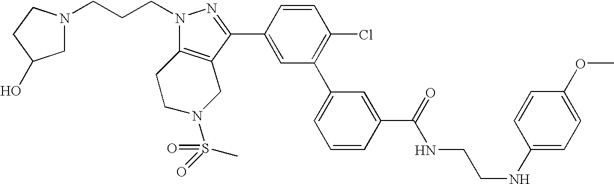

### Example 28

### 2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid [2-(4-methoxy-phenylamino)-ethyl]-amide

The title compound was prepared according to the methods described for Intermediate 1 and Example 1, substituting 2-pyrrolidinol for pyrrolidine in Intermediate 1, Step D. HPLC: Rₜ=1.375. MS (ESI): mass calcd. for C₃₆H₄₃ClN₆O₅S, 706.3; m/z found, 707.3 [M+H]⁺.

The compounds in Example 29-30 were prepared according to the methods described for Intermediate 1 and Example 1, substituting 2-pyrrolidinol for pyrrolidine in Intermediate 1, Step D, omitting Example 1, Step A, and substituting the appropriate amine for N¹-(4-methoxy-phenyl)-ethane-1,2-diamine in Example 1, Step B.

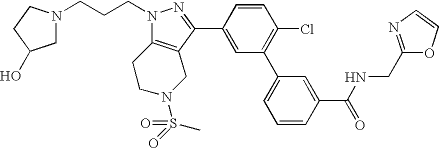

### Example 29

### 2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (oxazol-2-ylmethyl)-amide

HPLC: Rₜ=1.415. MS (ESI): mass calcd. for C₃₁H₃₅ClN₆O₅S, 638.2; m/z found, 639.2 [M+H]⁺.

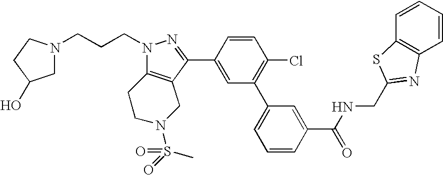

### Example 30

### 2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (benzothiazol-2-ylmethyl)-amide

HPLC: Rₜ=1.579. MS (ESI): mass calcd. for C₃₅H₃₇ClN₆O₄S₂, 704.2; m/z found, 705.2 [M+H]⁺.

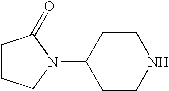

Intermediate 2; 1-Piperidin-4-yl-pyrrolidin-2-one.

A. 1-(1-Benzyl-piperidin-4-yl)-pyrrolidin-2-one. To a mechanically-stirring heterogeneous mixture of 1-benzyl-4-piperidone (5.0 g, 27.1 mmol) and ethyl-4-aminobutyrate hydrochloride (5 g, 32.5 mmol) in anhydrous dichloroethane (100 mL) was added NaBH(OAc)₃ (7.5 g, 35.2 mmol) portion-wise over 15 min. The resultant solution was stirred for 20 min at rt and was then treated with TEA (13.4 mL, 135.6 mmol) dropwise over 5 min. The resulting mixture was heated at 60° C. for 4 h. The mixture was cooled to rt, quenched by the slow addition of H₂O (100 mL), and extracted with CH₂Cl₂ (3×50 mL). The combined organic layers were dried over MgSO₄ and concentrated. The residue was partitioned between 5% EtOAc/hexanes and 1 N HCl (3×50 mL). The combined aqueous layers were adjusted to pH˜11 with aq. NaOH and extracted with EtOAc (3×50 mL). The organic layers were combined, washed with brine, dried over MgSO₄, and concentrated. Recrystallization using 5% EtOAc in hexanes gave the desired white solid (6.24 g, 89%).

B. 1-Piperidin-4-yl-pyrrolidin-2-one. A Parr bottle containing a solution of the above compound (4.8 g, 18.7 mmol) in absolute EtOH (35 ml) and 10 wt. % Pd/C (0.48 g) was stoppered and placed on a Parr shaker under H₂ (45 psi). After shaking for 36 h, the mixture was filtered through a pad of diatomaceous earth, washing with EtOAc. The filtrate was concentrated. The residue was diluted with warm Et₂O and filtered to remove insoluble particulates. The filtrate was concentrated to yield the desired product (2.8 g, 90%). HPLC: Rₜ=0.128. MS (ESI): mass calcd. for C₉H₁₆N₂O, 168.1; m/z found, 169.1 [M+H]⁺.

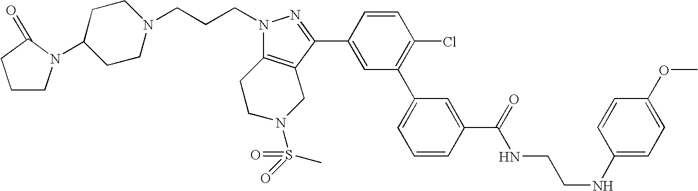

### Example 31

### 2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(4-methoxy-phenylamino)-ethyl]-amide

The title compound was prepared according to the methods described for Intermediate 1 and Example 1, substituting Intermediate 2 for pyrrolidine in Intermediate 1, Step D. HPLC: Rₜ=1.361. MS (ESI): mass calcd. for C₄₁H₅₀ClN₇O₅S, 787.3; m/z found, 788.3 [M+H]⁺.

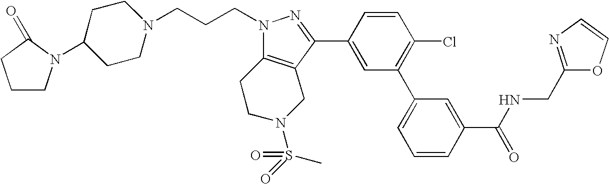

### Example 32

### 2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (oxazol-2-ylmethyl)-amide

The title compound was prepared according to the methods described for Intermediate 1 and Example 1, substituting Intermediate 2 for pyrrolidine in Intermediate 1, Step D, omitting Example 1, Step A, and substituting oxazol-2-yl-methylamine for N¹-(4-methoxy-phenyl)-ethane-1,2-diamine in Example 1, Step B. HPLC: Rₜ=1.376. MS (ESI): mass calcd. for C₃₆H₄₂ClN₇O₅S, 719.3; m/z found, 720.3 [M+H]⁺.

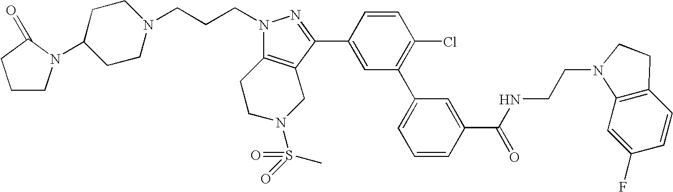

### Example 33

### 2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(6-fluoro-2,3-dihydro-indol-1-yl)-ethyl]-amide

The title compound was prepared according to the methods described for Intermediate 1 and Example 8, substituting Intermediate 2 for pyrrolidine in Intermediate 1, Step D and the appropriate indole for 5-fluoro-2,3-dihydro-(1H)-indole in Example 8, Step A. HPLC: Rₜ=1.580. MS (ESI): mass calcd. for C₄₂H₄₉ClFN₇O₄S, 801.3; m/z found, 802.3 [M+H]⁺.

The compounds in Example 34-35 were prepared according to the methods described for Intermediate 1 and Example 1, substituting Intermediate 2 for pyrrolidine in Intermediate 1, Step D, omitting Example 1, Step A, and substituting the appropriate amine for N¹-(4-methoxy-phenyl)-ethane-1,2-diamine in Example 1, Step B.

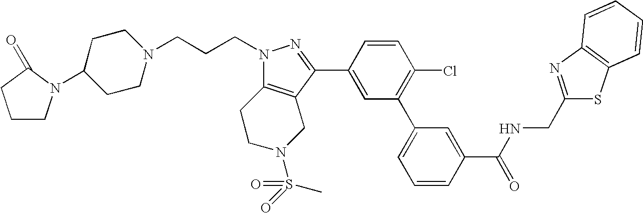

### Example 34

### 2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (benzothiazol-2-ylmethyl)-amide

HPLC: Rₜ=1.582. MS (ESI): mass calcd. for C₄₀H₄₄ClN₇O₄S₂, 785.3; m/z found, 786.2 [M+H]⁺.

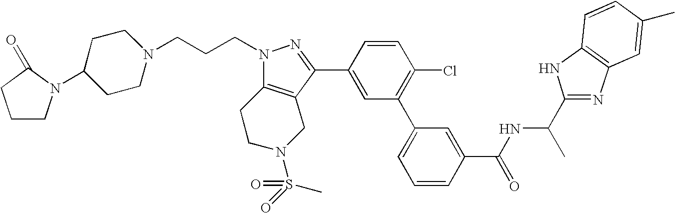

### Example 35

### 2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [1-(5-methyl-1H-benzoimidazol-2-yl)-ethyl]-amide

HPLC: Rₜ=1.402. MS (ESI): mass calcd. for C₄₂H₄₉ClN₈O₄S, 796.3; m/z found, 797.3 [M+H]⁺.

The compounds in Example 36-41 were prepared according to the methods described for Intermediate 1 and Example 1, omitting Intermediate 1, Step A, substituting 1-acetyl-piperidin-4-one for piperidone in Intermediate 1, Step B, omitting Example 1, Step A, and substituting the appropriate amine for N¹-(4-methoxy-phenyl)-ethane-1,2-diamine in Example 1, Step B.

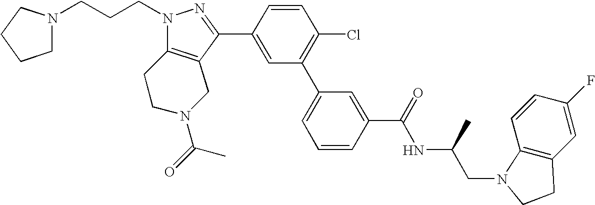

### Example 36

### (S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-fluoro-2,3-dihydro-indol-1-yl)-1-methyl-ethyl]-amide

HPLC: Rₜ=1.555. MS (ESI): mass calcd. for C₃₉H₄₄ClFN₆O₂, 682.3; m/z found, 682.0 [M]⁺.

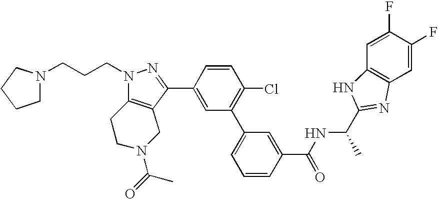

### Example 37

### (S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5,6-difluoro-1H-benzoimidazol-2-yl)-ethyl]-amide

HPLC: Rₜ=1.407. MS (ESI): mass calcd. for C₃₇H₃₈ClF₂N₇O₂, 685.3; m/z found, 686.3 [M+H]⁺.

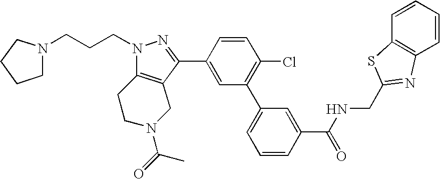

### Example 38

### 5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid (benzothiazol-2-ylmethyl)-amide

HPLC: Rₜ=1.688. MS (ESI): mass calcd. for C₃₆H₃₇ClN₆O₂S, 652.24; m/z found, 653.2 [M+H]⁺.

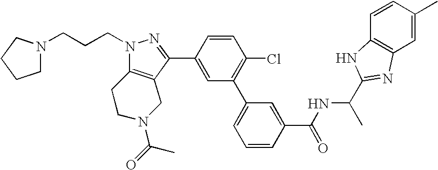

### Example 39

### 5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5-methyl-1H-benzoimidazol-2-yl)-ethyl]-amide

HPLC: Rₜ=1.403. MS (ESI): mass calcd. for C₃₈H₄₂ClN₇O₂, 663.3; m/z found, 664.3 [M+H]⁺.

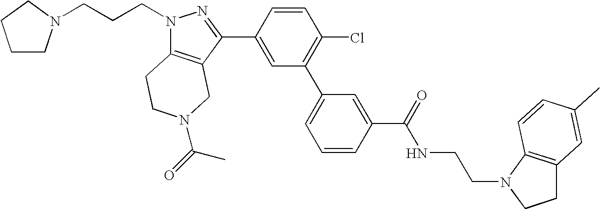

### Example 40

### 5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-methyl-2,3-dihydro-indol-1-yl)-ethyl]-amide

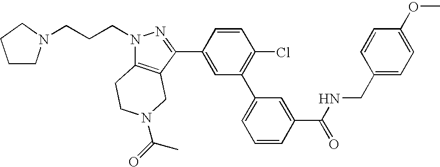

### Example 41

### 5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid 4-methoxy-benzylamide

HPLC: Rₜ=1.568. MS (ESI): mass calcd. for C₃₆H₄₀ClN₅O₃, 625.3; m/z found, 626.3 [M+H]⁺.

The compounds in Example 42-47 were prepared according to the methods described for the immediately preceding examples, with the appropriate substituent changes.

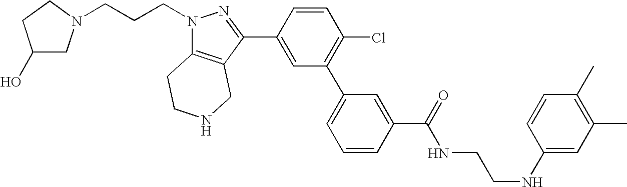

### Example 42

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(3,4-dimethyl-phenylamino)-ethyl]-amide

HPLC: Rₜ=1.387. MS (ESI): mass calcd. for C₃₆H₄₃ClN₆O₂, 626.3; m/z found, 627.3 [M+H]⁺.

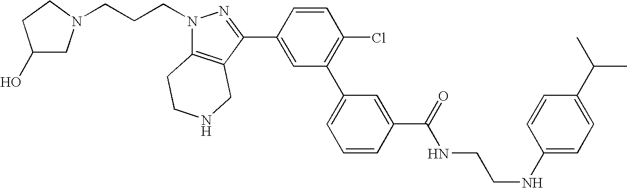

### Example 43

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-isopropyl-phenylamino)-ethyl]-amide

HPLC: Rₜ=1.448. MS (ESI): mass calcd. for C₃₇H₄₅ClN₆O₂, 640.3; m/z found, 641.3 [M+H]⁺.

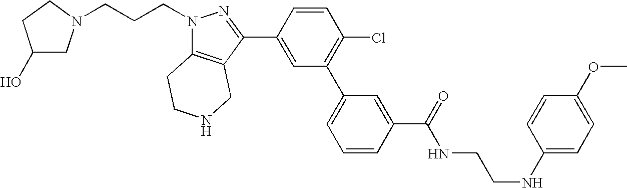

### Example 44

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-methoxy-phenylamino)-ethyl]-amide

HPLC: Rₜ=1.325. MS (ESI): mass calcd. C₃₅H₄₁ClN₆O₃, 628.3; m/z found, 628.3 [M]⁺.

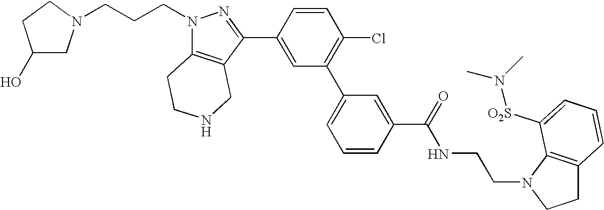

### Example 45

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(7-dimethylsulfamoyl-2,3-dihydro-indol-1-yl)-ethyl]-amide

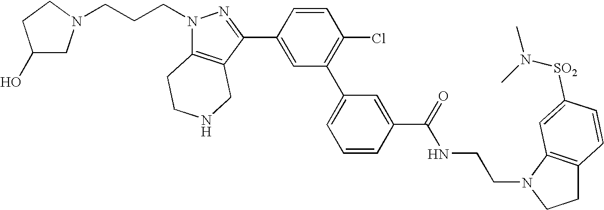

### Example 46

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(6-dimethylsulfamoyl-2,3-dihydro-indol-1-yl)-ethyl]-amide

HPLC: Rₜ=1.487. MS (ESI): mass calcd. for C₃₈H₄₆ClN₇O₄S, 731.3; m/z found, 732.3 [M+H]⁺.

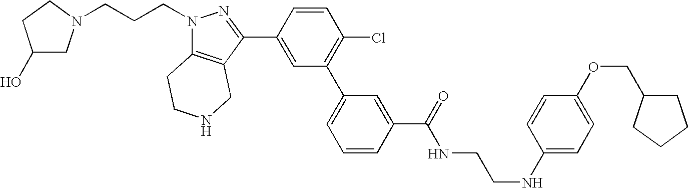

### Example 47

### 2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-cyclopentylmethoxy-phenylamino)-ethyl]-amide

HPLC: Rₜ=1.561. MS (ESI): mass calcd. for C₄₀H₄₉ClN₆O₃, 696.36; m/z found, 697.3 [M+H]⁺.

### Biological Testing:

Human CatS was cloned into pFB+HT (from Stratagene clone), expressed in Sf9 cells, and purified over a Ni column. Fractions were concentrated and activated at pH 4.0 for 6 hr and then purified over a thiopropylsepharose column and eluted with 1×TBS, 500 mM NaCl, 1 mM EDTA, 25 mM DTT pH 7.6. Glycerol (50%) was added in a 1:1 (vol/vol) ratio (25% glycerol final) and protein was stored at −80° C. in 5 μL aliquots. Compounds were tested for their ability to inhibit CatS hydrolysis of the fluorescent substrate Z-Val-Val-Arg-AFC (catalog #I-1540, Bachem). Inhibitor solutions in DMSO were serially diluted and mixed with a solution of substrate in 150 mM sodium acetate pH 5.0 containing 1.5 mM DTT and 150 mM NaCl (optionally also containing 0.005% Triton X-100), which yielded a final substrate concentration of 10 μM and DMSO concentration of 1%. Reactions were initiated by the addition of CatS (1.5 nM final concentration of active enzyme; determined by titration against a tight-binding known standard inhibitor [Kᵢ=35 nM] using Equation 1 below) and brief mixing. The increase in fluorescence over time was monitored using λ_{excitation}=400 nm and λₑₘᵢₛₛᵢₒₙ=505 nm. Initial rates were fit to the Morrison equation (Williams, J. W.; Morrison, J. F. The Kinetics of Reversible Tight-Binding Inhibition. Methods in Enzymology 1979, 63, 437-467) and apparent Kᵢ (Kᵢ^{app}) determined using Graphpad Prism software.

$$v = {v}_{0} \cdot \frac{{[ E ]}_{0} - {[ I ]}_{0} - {K}_{i}^{app} + \sqrt{{( {[ E ]}_{0} - {[ I ]}_{0} - {K}_{i}^{app} )}^{2} + {4 [ E ]}_{0} {K}_{i}^{app}}}{{2 [ E ]}_{0}} \qquad ( 1 )$$

In equation 1, v is the initial rate measured in the presence of [I]₀, the inhibitor concentration, using an enzyme concentration [E]₀. v₀ is the initial rate measured in the absence of inhibitor. Kᵢ^{app} values are given in Table 1.

TABLE 1

| EX. | CatS Kᵢ^{app} (μM) |
|---|---|
| 1 | 0.08497 |
| 2 | 0.3445 |
| 3 | 0.3349 |
| 4 | 0.2532 |
| 5 | 0.06022 |
| 6 | 0.3167 |
| 7 | 0.8759 |
| 8 | 0.2373 |
| 9 | 0.1376 |
| 10 | 0.04936 |
| 11 | 0.1348 |
| 12 | 0.2397 |
| 13 | 0.33 |
| 14 | 0.35 |
| 15 | 0.45 |
| 16 | 0.02396 |
| 17 | 0.1249 |
| 18 | 0.02257 |
| 19 | 0.2686 |
| 20 | 0.1879 |
| 21 | 0.07028 |
| 22 | 0.0754 |
| 23 | 0.08887 |
| 24 | 0.1751 |
| 25 | 0.1167 |
| 26 | 0.7125 |
| 27 | 1.393 |
| 28 | 0.01954 |
| 29 | 0.4772 |
| 30 | 0.3247 |
| 31 | 0.02726 |
| 32 | 0.2641 |
| 33 | 0.1633 |
| 34 | 0.07228 |
| 35 | 0.1232 |
| 36 | 0.736 |
| 37 | 0.03848 |
| 38 | 0.2306 |
| 39 | 0.03698 |
| 40 | 0.2283 |
| 41 | 0.4299 |
| 42 | 0.04165 |
| 43 | 0.04854 |
| 44 | 0.03042 |
| 45 | 0.2254 |
| 46 | 0.5096 |
| 47 | 0.05367 |

While the invention has been illustrated by reference to examples, it is understood that the invention is intended not to be limited to the foregoing detailed description.

What is claimed is:

1. A compound of Formula (I): 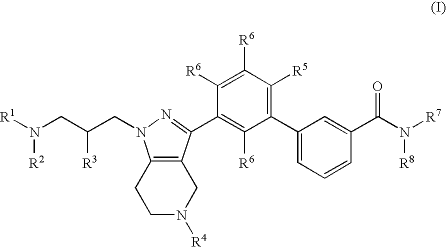
wherein:
R¹ and R² taken together with the nitrogen to which they are attached form a saturated monocyclic heterocycloalkyl group, optionally containing one additional heteroatom ring member that is O, S, or NR^{a}, and being unsubstituted or substituted with one, two, or three R^{b} substituents;
where R^{a} is H, C₁₋₄alkyl, —COC₁₋₄alkyl, or —CO₂C₁₋₄alkyl;
each R^{b} substituent is independently:
i) OH, C₁₋₄alkyl, CF₃, NR^{c}R^{d}, —COC₁₋₄alkyl, —CO₂C₁₋₄alkyl; or —CONR^{e}R^{f};
ii) a monocyclic heterocycloalkyl group unsubstituted or substituted with C₁₋₄alkyl, OH, —OC₁₋₄alkyl, NR^{c}R^{d}, or halo; or
iii) a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
where R^{c} is H or C₁₋₄alkyl;
R^{d} is H, C₁₋₄alkyl, —COC₁₋₄alkyl, —CO₂C₁₋₄alkyl, or —CONR^{x}R^{y};
where R^{x} and R^{y} are each independently H or C₁₋₄alkyl; and
R^{e} and R^{f} are each independently H or C₁₋₄alkyl;
or, alternatively, two R^{b} substituents at the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
R³ is H, OH, C₁₋₄alkyl, or —OC₁₋₄alkyl;
R⁴ is H, C₁₋₄alkyl, —COC₁₋₄alkyl, —SO₂C₁₋₄alkyl, —SO₂CF₃, —CONH₂, —CONHC₁₋₄alkyl, —CON(C₁₋₄alkyl)₂, —COCO₂C₁₋₄alkyl, —COCONH₂, or —COCONHC₁₋₄alkyl;
R⁵ is halo or CF₃;
each R⁶ is H or F;
R⁷ is H or C₁₋₆alkyl; and
R⁸ is Ar, —CH(Rⁱ)Ar, 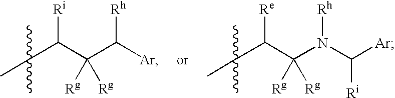
where each R^{g} is H or C₁₋₄alkyl, or two R^{g} groups together form a carbonyl;
each R^{h} is H or C₁₋₄alkyl;
Rⁱ is H or C₁₋₄alkyl; and
Ar is a phenyl, naphthyl, monocyclic heteroaryl, or bicyclic heteroaryl group, unsubstituted or substituted with one, two, or three R^{j} substituents;
where each R^{j} substituent is independently selected from the group consisting of:
C₁₋₄alkyl, monocyclic cycloalkyl, phenyl, —OC₁₋₄alkyl, —O—(CH₂)₀₋₁-(monocyclic cycloalkyl), halo, CF₃, —COC₁₋₄alkyl, —CO₂C₁₋₄alkyl, CO₂H, CN, NR^{r}R^{s}, —N(R^{r})COC₁₋₄alkyl, —N(R^{r})SO₂C₁₋₄alkyl, —NO₂, —SO₂C₁₋₄alkyl, —SO₂NR^{r}R^{s}, or —SO₃H, or two adjacent R^{j} substituents together form —(CH₂)₃—;
or, alternatively, an R^{j} substituent taken together with R^{h} forms —CH₂CH₂—;
where R^{r} and R^{s} are each independently H or C₁₋₄alkyl;
and pharmaceutically acceptable salts, prodrugs, and metabolites thereof.

2. A compound as defined in claim 1, wherein —NR¹R² is a structure of Formula (II): 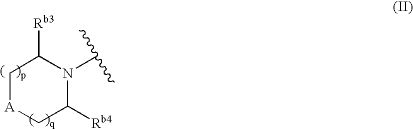
wherein:
A is NR^{a}, O, S, or C(R^{b1})(R^{b2});
where R^{a} is H or C₁₋₄alkyl;
R^{b1} is H, OH, or C₁₋₄alkyl; and
R^{b2} is H; a monocyclic heterocycloalkyl group unsubstituted or substituted with C₁₋₄alkyl, OH, —OC₁₋₄alkyl, NR^{c}R^{d}, or halo; or a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
one of R^{b3} and R^{b4} is H and the other is C₁₋₄alkyl;
p is 0, 1, or 2; and
q is 0, 1, 2, or 3;
with the proviso that when A is NR^{a}, O, S, or SO₂, then p and q are each greater than or equal to 1.

3. A compound as defined in claim 1, wherein R¹ and R² taken together with the nitrogen to which they are attached form azetidine, pyrrolidine, piperidine, piperazine substituted with R^{a}, morpholine, or thiomorpholine, each unsubstituted or substituted with one, two, or three R^{b} substituents.

4. A compound as defined in claim 1, wherein R¹ and R² taken together with the nitrogen to which they are attached form pyrrolidine or piperidine, each unsubstituted or substituted with one, two, or three R^{b} substituents.

5. A compound as defined in claim 1, wherein R^{a} is H, methyl, isopropyl, acetyl, or tert-butoxycarbonyl.

6. A compound as defined in claim 1, wherein each R^{b} substituent is independently OH, methyl, propyl, CF₃, dimethylamino, acetamido, tert-butoxycarbamoyl, fluoro, or methoxy.

7. A compound as defined in claim 1, wherein each R^{b} substituent is independently pyrrolidinyl, 2-oxo-pyrrolidinyl, or piperidinyl.

8. A compound as defined in claim 1, wherein each R^{b} substituent is independently pyrrolidin-1-yl or 2-oxo-pyrrolidin-1-yl.

9. A compound as defined in claim 1, wherein R³ is H or OH.

10. A compound as defined in claim 1, wherein R⁴ is H, methyl, —SO₂CH₃, acetyl, or tert-butoxycarbonyl.

11. A compound as defined in claim 1, wherein R⁴ is —SO₂CH₃.

12. A compound as defined in claim 1, wherein R⁵ is chloro or CF₃.

13. A compound as defined in claim 1, wherein R⁵ is chloro.

14. A compound as defined in claim 1, wherein R⁶ is H.

15. A compound as defined in claim 1, wherein R⁷ is H.

16. A compound as defined in claim 1, wherein R⁸ is Ar.

17. A compound as defined in claim 1, wherein R⁸ is —CH(Rⁱ)Ar.

18. A compound as defined in claim 1, wherein R⁸ is —(CH₂)₂N(R^{h})Ar.

19. A compound as defined in claim 1, wherein each R^{g} is H or methyl.

20. A compound as defined in claim 1, wherein R^{h} is H or methyl.

21. A compound as defined in claim 1, wherein Rⁱ is H, methyl, or ethyl.

22. A compound as defined in claim 1, wherein Ar is a phenyl, naphthyl, pyridinyl, pyrimidinyl, oxazolyl, thiophenyl, thiazolyl, indanyl, indolyl, benzimidazolyl, or benzothiazolyl group, unsubstituted or substituted with one, two, or three R^{j} substituents.

23. A compound as defined in claim 1, wherein Ar is 4-methoxyphenyl, 4-methylphenyl, indan-4-yl, 3-chloro-4-methoxyphenyl, 4-cyclopentylmethoxy-phenyl, 6-methoxy-pyridin-3-yl, pyridin-3-yl, oxazol-2-yl, 1H-indol-2-yl, thiophen-2-yl, 5-methyl-1H-benzoimidazol-2-yl, 1H-benzoimidazol-2-yl, thiazol-2-yl, 5-chloro-1H-benzoimidazol-2-yl, 4-tert-butyl-thiazol-2-yl, 4-phenyl-thiazol-2-yl, 5-fluoro-benzothiazol-2-yl, benzothiazol-2-yl, 5,6-difluoro-1H-benzoimidazol-2-yl, 3,4-dimethyl-phenyl, or 4-isopropyl-phenyl.

24. A compound as defined in claim 1, wherein —N(R^{h})—Ar is 2,3-dihydro-indolyl, unsubstituted or substituted with one or two additional R^{j} substituents.

25. A compound as defined in claim 1, wherein —N(R^{h})—Ar is 5-fluoro-2,3-dihydro-indol-1-yl, 7-dimethylsulfamoyl-2,3-dihydro-indol-1-yl, 6-dimethylsulfamoyl-2,3-dihydro-indol-1-yl, 6-fluoro-2,3-dihydro-indol-1-yl, or 5-methyl-2,3-dihydro-indol-1-yl.

26. A compound as defined in claim 1, wherein each R^{j} substituent is independently methyl, isopropyl, tert-butyl, cyclopentyl, phenyl, methoxy, isopropoxy, cyclopentylmethoxy, cyclohexyloxy, chloro, fluoro, CF₃, —NO₂, —SO₂N(CH₃)₂, or —SO₃H, or two adjacent R^{j} substituents together form —(CH₂)₃—.

27. A compound as defined in claim 1, wherein an R^{j} substituent taken together with R^{h} forms —CH₂CH₂—.

28. A compound selected from the group consisting of:
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (2-p-tolylamino-ethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(indan-4-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-chloro-4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-cyclopentylmethoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-methoxy-pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-ethyl]-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-1-methyl-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(7-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (oxazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (1H-indol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiophen-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 4-methoxy-benzylamide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-propyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiazol-2-ylmethyl)-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-chloro-1H-benzoimidazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-tert-butyl-thiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-phenyl-thiazol-2-ylmethyl)-amide;
(R)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(4-phenyl-thiazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-fluoro-benzothiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [(4-methoxy-phenylcarbamoyl)-methyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-methoxy-benzylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (oxazol-2-ylmethyl)-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(4-methoxy-phenylamino)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (oxazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(6-fluoro-2,3-dihydro-indol-1-yl)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid
(benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [1-(5-methyl-1H-benzoimidazol-2-yl)-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-fluoro-2,3-dihydro-indol-1-yl)-1-methyl-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5,6-difluoro-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid (benzothiazol-2-ylmethyl)-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5-methyl-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-methyl-2,3-dihydro-indol-1-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid 4-methoxy-benzylamide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(3,4-dimethyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-isopropyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(7-dimethylsulfamoyl-2,3-dihydro-indol-1-yl)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(6-dimethylsulfamoyl-2,3-dihydro-indol-1-yl)-ethyl]-amide; and
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-cyclopentylmethoxy-phenylamino)-ethyl]-amide;
and pharmaceutically acceptable salts thereof.

29. A compound as defined in claim 1, wherein said compound is a compound of Formula (I) or a pharmaceutically acceptable salt of a compound of Formula (I).

30. A pharmaceutical composition for treating a disease, disorder, or medical condition mediated by cathepsin S activity, comprising:
(a) an effective amount of at least one chemical entity selected from compounds of Formula (I): 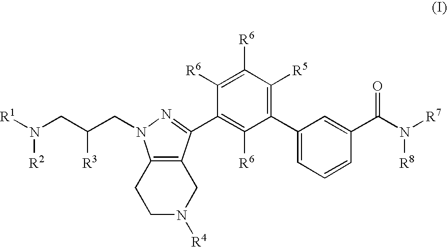
wherein:
R¹ and R² taken together with the nitrogen to which they are attached form a saturated monocyclic heterocycloalkyl group, optionally containing one additional heteroatom ring member that is O, S, or NR^{a}, and being unsubstituted or substituted with one, two, or three R^{b} substituents;
where R^{a} is H, C₁₋₄alkyl, —COC₁₋₄alkyl, or —CO₂C₁₋₄alkyl;
each R^{b} substituent is independently:
i) OH, C₁₋₄alkyl, CF₃, NR^{c}R^{d}, —COC₁₋₄alkyl, —CO₂C₁₋₄alkyl; or —CONR^{e}R^{f};
ii) a monocyclic heterocycloalkyl group unsubstituted or substituted with C₁₋₄alkyl, OH, —OC₁₋₄alkyl, NR^{c}R^{d}, or halo; or
iii) a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
where R^{c} is H or C₁₋₄alkyl;
R^{d} is H, C₁₋₄alkyl, —COC₁₋₄alkyl, —CO₂C₁₋₄alkyl, or —CONR^{x}R^{y};
where R^{x} and R^{y} are each independently H or C₁₋₄alkyl; and
R^{e} and R^{f} are each independently H or C₁₋₄alkyl;
or, alternatively, two R^{b} substituents at the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
R³ is H, OH, C₁₋₄alkyl, or —OC₁₋₄alkyl;
R⁴ is H, C₁₋₄alkyl, —COC₁₋₄alkyl, —SO₂C₁₋₄alkyl, —SO₂CF₃, —CONH₂, —CONHC₁₋₄alkyl, —CON(C₁₋₄alkyl)₂, —COCO₂C₁₋₄alkyl, —COCONH₂, or —COCONHC₁₋₄alkyl;
R⁵ is halo or CF₃;
each R⁶ is H or F;
R⁷ is H or C₁₋₆alkyl; and
R⁸ is Ar, —CH(Rⁱ)Ar, 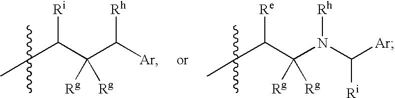
where each R^{g} is H or C₁₋₄alkyl, or two R^{g} groups together form a carbonyl;
each R^{h} is H or C₁₋₄alkyl;
Rⁱ is H or C₁₋₄alkyl; and
Ar is a phenyl, naphthyl, monocyclic heteroaryl, or bicyclic heteroaryl group, unsubstituted or substituted with one, two, or three R^{j} substituents;
where each R^{j} substituent is independently selected from the group consisting of:
C₁₋₄alkyl, monocyclic cycloalkyl, phenyl, —OC₁₋₄alkyl, —O—(CH₂)₀₋₁-(monocyclic cycloalkyl), halo, CF₃, —COC₁₋₄alkyl, —CO₂C₁₋₄alkyl, CO₂H, CN, NR^{r}R^{s}, —N(R^{r})COC₁₋₄alkyl, —N(R^{r})SO₂C₁₋₄alkyl, —NO₂, —SO₂C₁₋₄alkyl, —SO₂NR^{r}R^{s}, or —SO₃H, or two adjacent R^{j} substituents together form —(CH₂)₃—;
or, alternatively, an R^{j} substituent taken together with R^{h} forms —CH₂CH₂—;
where R^{r} and R^{s} are each independently H or C₁₋₄alkyl;
and pharmaceutically acceptable salts, prodrugs, and metabolites thereof; and
(b) a pharmaceutically acceptable excipient.

31. A pharmaceutical composition according to claim 30, wherein said chemical entity is selected from the group consisting of:
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (2-p-tolylamino-ethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(indan-4-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-chloro-4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-cyclopentylmethoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-methoxy-pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-ethyl]-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-1-methyl-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(7-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (oxazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (1H-indol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiophen-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 4-methoxy-benzylamide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-propyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiazol-2-ylmethyl)-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-chloro-1H-benzoimidazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-tert-butyl-thiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-phenyl-thiazol-2-ylmethyl)-amide;
(R)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(4-phenyl-thiazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-fluoro-benzothiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [(4-methoxy-phenylcarbamoyl)-methyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-methoxy-benzylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (oxazol-2-ylmethyl)-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(4-methoxy-phenylamino)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (oxazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(6-fluoro-2,3-dihydro-indol-1-yl)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid
(benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [1-(5-methyl-1H-benzoimidazol-2-yl)-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-fluoro-2,3-dihydro-indol-1-yl)-1-methyl-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5,6-difluoro-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid (benzothiazol-2-ylmethyl)-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5-methyl-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-methyl-2,3-dihydro-indol-1-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid 4-methoxy-benzylamide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(3,4-dimethyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-isopropyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(7-dimethylsulfamoyl-2,3-dihydro-indol-1-yl)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(6-dimethylsulfamoyl-2,3-dihydro-indol-1-yl)-ethyl]-amide; and
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-cyclopentylmethoxy-phenylamino)-ethyl]-amide;
and pharmaceutically acceptable salts thereof.

32. A method of treating a subject suffering from or diagnosed with a disease, disorder, or medical condition mediated by cathepsin S activity, comprising administering to a subject in need of such treatment an effective amount of at least one chemical entity selected from compounds of Formula (I): 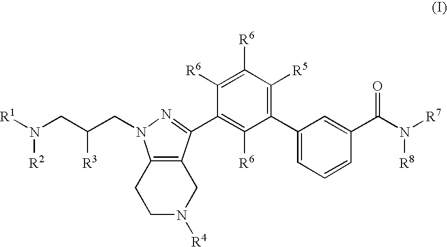
wherein:
R¹ and R² taken together with the nitrogen to which they are attached form a saturated monocyclic heterocycloalkyl group, optionally containing one additional heteroatom ring member that is O, S, or NR^{a}, and being unsubstituted or substituted with one, two, or three R^{b} substituents;
where R^{a} is H, C₁₋₄alkyl, —COC₁₋₄alkyl, or —CO₂C₁₋₄alkyl;
each R substituent is independently:
i) OH, C₁₋₄alkyl, CF₃, NR^{c}R^{d}, —COC₁₋₄alkyl, —CO₂C₁₋₄alkyl; or —CONR^{e}R^{f};
ii) a monocyclic heterocycloalkyl group unsubstituted or substituted with C₁₋₄alkyl, OH, —OC₁₋₄alkyl, NR^{c}R^{d}, or halo; or
iii) a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
where R^{c} is H or C₁₋₄alkyl;
R^{d} is H, C₁₋₄alkyl, —COC₁₋₄alkyl, —CO₂C₁₋₄alkyl, or —CONR^{x}R^{y};
where R^{x} and R^{y} are each independently H or C₁₋₄alkyl; and
R^{e} and R^{f} are each independently H or C₁₋₄alkyl;
or, alternatively, two R^{b} substituents at the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
R³ is H, OH, C₁₋₄alkyl, or —OC₁₋₄alkyl;
R⁴ is H, C₁₋₄alkyl, —COC₁₋₄alkyl, —SO₂C₁₋₄alkyl, —SO₂CF₃, —CONH₂, —CONHC₁₋₄alkyl, —CON(C₁₋₄alkyl)₂, —COCO₂C₁₋₄alkyl, —COCONH₂, or —COCONHC₁₋₄alkyl;
R⁵ is halo or CF₃;
each R⁶ is H or F;
R⁷ is H or C₁₋₆alkyl; and
R⁸ is Ar, —CH(Rⁱ)Ar, 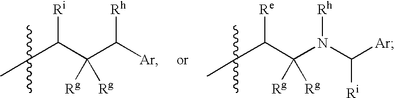
where each R^{g} is H or C₁₋₄alkyl, or two R^{g} groups together form a carbonyl;
each R^{h} is H or C₁₋₄alkyl;
Rⁱ is H or C₁₋₄alkyl; and
Ar is a phenyl, naphthyl, monocyclic heteroaryl, or bicyclic heteroaryl group, unsubstituted or substituted with one, two, or three R^{j} substituents;
where each R^{j} substituent is independently selected from the group consisting of:
C₁₋₄alkyl, monocyclic cycloalkyl, phenyl, —OC₁₋₄alkyl, —O—(CH₂)₀₋₁-(monocyclic cycloalkyl), halo, CF₃, —COC₁₋₄alkyl, —CO₂C₁₋₄alkyl, CO₂H, CN, NR^{r}R^{s}, —N(R^{r})COC₁₋₄alkyl, —N(R^{r})SO₂C₁₋₄alkyl, —NO₂, —SO₂C₁₋₄alkyl, —SO₂NR^{r}R^{s}, or —SO₃H, or two adjacent R^{j} substituents together form —(CH₂)₃—;
or, alternatively, an R^{j} substituent taken together with R^{h} forms —CH₂CH₂—;
where R^{r} and R^{s} are each independently H or C₁₋₄alkyl;
and pharmaceutically acceptable salts, prodrugs, and metabolites thereof.

33. A method according to claim 32, wherein said chemical entity is selected from the group consisting of:
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (2-p-tolylamino-ethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(indan-4-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-chloro-4-methoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(4-cyclopentylmethoxy-phenylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-methoxy-pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(pyridin-3-ylamino)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-ethyl]-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(5-fluoro-2,3-dihydro-indol-1-yl)-1-methyl-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(7-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(6-dimethylsulfamoyl-2,3-dihydro-indol-1-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (oxazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (1H-indol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiophen-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid 4-methoxy-benzylamide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-propyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (thiazol-2-ylmethyl)-amide;
(S)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(5-methyl-1H-benzoimidazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-chloro-1H-benzoimidazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-tert-butyl-thiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (4-phenyl-thiazol-2-ylmethyl)-amide;
(R)-2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [1-(4-phenyl-thiazol-2-yl)-ethyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid (5-fluoro-benzothiazol-2-ylmethyl)-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [(4-methoxy-phenylcarbamoyl)-methyl]-amide;
2′-Chloro-5′-[5-methanesulfonyl-1-(3-pyrrolidin-1-yl-propyl)-4,5,6,7-tetrahydro-1H-pyrazolo[4,3-c]pyridin-3-yl]-biphenyl-3-carboxylic acid [2-(3-methoxy-benzylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (oxazol-2-ylmethyl)-amide;
2′-Chloro-5′-{1-[3-(3-hydroxy-pyrrolidin-1-yl)-propyl]-5-methanesulfonyl-4,5,6,7-tetrahydro-1H-pyrazolo[4,3-c]pyridin-3-yl}-biphenyl-3-carboxylic acid (benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(4-methoxy-phenylamino)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (oxazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [2-(6-fluoro-2,3-dihydro-indol-1-yl)-ethyl]-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid (benzothiazol-2-ylmethyl)-amide;
2′-Chloro-5′-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-biphenyl-3-carboxylic acid [1-(5-methyl-1H-benzoimidazol-2-yl)-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-fluoro-2,3-dihydro-indol-1-yl)-1-methyl-ethyl]-amide;
(S)-5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5,6-difluoro-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid (benzothiazol-2-ylmethyl)-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [1-(5-methyl-1H-benzoimidazol-2-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid [2-(5-methyl-2,3-dihydro-indol-1-yl)-ethyl]-amide;
5′-[5-Acetyl-1-(3-pyrrolidin-1-yl-propyl)-4,5,6,7-tetrahydro-1H-pyrazolo[4,3-c]pyridin-3-yl]-2′-chloro-biphenyl-3-carboxylic acid 4-methoxy-benzylamide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(3,4-dimethyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-isopropyl-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-methoxy-phenylamino)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(7-dimethylsulfamoyl-2,3-dihydro-indol-1-yl)-ethyl]-amide;
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(6-dimethylsulfamoyl-2,3-dihydro-indol-1-yl)-ethyl]-amide; and
2′-Chloro-5′-{1-[3-(3-hydroxy-pyrrolidin-1-yl)-propyl]-4,5,6,7-tetrahydro-1H-pyrazolo[4,3-c]pyridin-3-yl}-biphenyl-3-carboxylic acid [2-(4-cyclopentylmethoxy-phenylamino)-ethyl]-amide;
and pharmaceutically acceptable salts thereof.

34. A method according to claim 32, wherein the disease, disorder, or medical condition is an autoimmune disease, an allergic condition, inflammation, a bowel disorder, tissue transplant rejection, pain, or cancer.

35. A method according to claim 32, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

36. A method according to claim 32, wherein the disease, disorder, or medical condition is selected from the group consisting of: psoriasis, pain, multiple sclerosis, atherosclerosis, and rheumatoid arthritis.
